(19) United States
(12) Patent Application Publication
(10) Pub. No.: US 2024/0309468 A1
(43) Pub. Date: Sep. 19, 2024

(54) ORAL SWAB-BASED TEST FOR THE DETECTION OF VARIOUS DISEASE STATES IN DOMESTIC CATS

(71) Applicant: BASEPAWS, Torrance, CA (US)

(72) Inventors: Damian Kao, Playa Vista, CA (US); Yuliana Mihaylova, Playa Vista, CA (US)

(21) Appl. No.: 18/578,291

(22) PCT Filed: Jul. 14, 2022
(86) PCT No.: PCT/US22/73742

§ 371 Date: Jan. 10, 2024

Related U.S. Application Data
(60) Provisional application No. 63/221,559, filed on Jul. 14, 2021.

(51) Int. Cl. C12Q 1/689; C12Q 1/6806; G16B 30/10; G16H 50/20; G16H 50/30

(52) U.S. Cl. CPC C12Q 1/689; C12Q 1/6806; G16B 30/10; G16H 50/20; G16H 50/30

(57) ABSTRACT

Systems and methods for screening for, indicating, diagnosing, treating, and identifying dermatologic and/or respiratory disease states in domestic cats. Systems and methods for screening for, indicating, diagnosing, treating, and identifying dermatologic and/or respiratory disease states in domestic cats. Systems and methods for screening for, indicating, diagnosing, treating, and identifying dermatologic and/or respiratory disease states in domestic cats.

### CROSS-REFERENCE TO RELATED APPLICATIONS

This application is a nationalization of PCT/US22/73742, filed Jul. 14, 2022, which claims the benefit of and priority to U.S. Provisional Application No. 63/221,559, filed Jul. 14, 2021. The entire contents of each of the foregoing are incorporated herein by specific reference.

### BACKGROUND

### Technical Field

This disclosure relates to systems and methods for screening for, detecting, diagnosing, treating, and identifying dermatologic and respiratory disease states in domestic cats.

### Related Technology

In recent years, the connection between the microbial composition of different body sites (the microbiome) and disease has gained more prominence in biomedical research. It is often debated whether the microbiome contributes to disease pathology or is simply a readout of a body's state. In most cases, the answer is unclear. It is also possible that the microbiota of different body sites may act in synergy or in opposition to cause/contribute to disease pathology. As a result, the microbiome holds promise from both a diagnostic and a treatment perspective.

It has been established that the oral cavity is second only to the lower gastrointestinal tract in terms of microbial diversity and, as such, has the potential to be directly relevant to a plethora of disease states. The human oral microbiome's importance has been implicated in dental as well as systemic diseases. Some examples include chronic inflammatory diseases, such as inflammatory bowel disease (IBD), rheumatoid arthritis, bacterial endocarditis, atherosclerosis, etc. Changes in the oral microbiome have also recently been discovered in people with allergic diseases, including atopic dermatitis.

While study of the human oral microbiome has advanced in recent years, the same cannot be said for the oral microbiome of companion animals and, in particular, cats. Cats suffer from a multitude of allergic dermatitis conditions and the connection between the oral microbiome and these conditions has not been investigated to date.

For example, atopic dermatitis is estimated to affect 12.5% of all cats, while around 1% of all feline vet visits are associated with food allergic dermatitis. Atopic dermatitis, food allergic dermatitis, flea allergic dermatitis and environmental allergies often present with similar symptoms, making it challenging to distinguish between them. The symptoms may include pruritus, scabbing and hair loss, but there is no consistent disease presentation from one case to the next. No single clinical diagnostic test can currently reliably distinguish between these four dermatologic conditions. Since most antigen-based pet allergen testing is limited and unreliable, a thorough approach to reaching an accurate diagnosis is based on exclusion and relies on performance of various tests: 8- to 12-week-long food trials where certain foods are removed then re-introduced to the diet; 8-week-long flea prevention treatment; and dermatophyte culture for ruling out fungal infections. Therefore, in most cases, it can be months before a definitive diagnosis is reached and appropriate treatment administered, thus prolonging animal suffering and decreasing their quality of life. This problem is also compounded by the fact that cat owners often notice the symptoms of dermatitis only after the cat has had the disease for a while, which can sometimes mean that secondary infections (often of bacterial or yeast origin) have developed as well, making the condition more persistent and difficult to treat.

Accordingly, there is a need for robust and accurate, yet safe, painless and affordable means for detecting and differentiating between feline dermatologic and inflammatory/respiratory diseases.

### SUMMARY

Embodiments of the present disclosure include computer systems, systems and methods for screening for, detecting, diagnosing, treating, and/or identifying dermatologic and/or respiratory disease states in cats. Using such tools to guide and complement veterinary health assessment can significantly improve dermatologic, respiratory, and allergic health outcomes, by leading to precise diagnosis and earlier implementation of treatment compared to relying on veterinary visits alone. Embodiments of the disclosed subject matter describe a method for interrogating the oral microbiome of a cat. The disclosed methods interrogate the oral microbiome to detect microbe compositional abundance trends that may be associated with dermatologic disease states in cats. Detecting, identifying and/or quantifying microbial compositional abundance trends enables a practitioner to screen for and/or indicate whether a cat has a particular dermatologic and/or respiratory disease. Detecting and identifying dermatologic and/or respiratory disease states enables the practitioner and/or the cat's owner to treat and/or prevent the dermatologic and/or respiratory disease state.

In some embodiments, a method is disclosed for detecting and/or indicating dermatologic and/or respiratory disease in cats. The method may include receiving an oral swab sample taken from a cat; manipulating the sample, such as heat treatment of the oral sample; and extracting microbial deoxyribonucleic acids (DNA) from the heat-treated sample. The method may additionally include sequencing the microbial DNA to identify which specific one or more microbes are present in the oral sample (and in what relative proportions), wherein identifying the specific one or more microbes enables generation of an oral microbial profile for the cat. The method may additionally include comparing the oral microbial profile for the cat against a reference database including defined microbial profiles, wherein the database identifies correlations between (i) profiles that include one or more microbes, and (ii) corresponding dermatologic and/or respiratory diseases; and based on a result of comparing the oral microbial profile against the database of defined microbial profiles, generating a risk score indicating a likelihood that the cat has a specific dermatologic and/or respiratory disease. In some embodiments, the dermatologic and/or respiratory disease state is selected from the group consisting of asthma, atopic dermatitis, flea allergic dermatitis, environmental allergic dermatitis, and food allergic dermatitis.

The method may further include treating the specific dermatologic and/or respiratory disease and/or administering a therapeutic treatment. In some embodiments, the therapeutic treatment may include administering a therapeutic compound, such as a compound designed to inhibit or encourage growth of a specific one or more microbes present in the oral microbiome of the cat. In some embodiments, the therapeutic compound includes a pre-biotic, a post-biotic, a pro-biotic, a medicament or a combination thereof. In some embodiments, the therapeutic compound may include an antibiotic, a corticosteroid, a bronchodilator, or a combination thereof. In some embodiments, the therapeutic treatment may include a topical treatment or bath to alleviate itching and promote healing of the skin. In some embodiments, the therapeutic treatment may include a dietary regimen which, in some cases, may be aimed at avoiding an allergen.

In some embodiments, a method for indicating dermatologic and/or respiratory diseases in cats includes receiving an oral swab sample taken from a cat and performing heat treatment on the oral sample. The method may also include performing magnetic beads-based deoxyribonucleic acid (DNA) extraction on the heat treated oral sample to extract microbial DNA that is present in the oral swab sample, and sequencing the microbial DNA to identify which specific one or more microbe(s) are present in the oral sample (and in what compositional abundance(s)), wherein identifying the specific one or more microbe(s) enables generation of an oral microbial profile for the cat. The method may additionally include comparing the oral microbial profile for the cat against a database of defined microbial profiles, wherein the database identifies correlations between (i) profiles that include one or more microbes (and their compositional abundance), and (ii) corresponding dermatologic and/or respiratory diseases; and based on a result of comparing the oral microbial profile against the database of defined microbial profiles, generating a risk score indicating a likelihood that the cat has a dermatologic and/or respiratory disease. The method may include, in response to generating the risk score and identifying the specific dermatologic and/or respiratory disease, administering a therapeutic treatment designed to treat the specific dermatologic and/or respiratory disease, recommending veterinary attention or follow-up examination, and/or recommending at-home care for the specific dermatologic and/or respiratory disease. The dermatologic and/or respiratory diseases are selected from the group consisting of asthma, atopic dermatitis, flea allergic dermatitis, environmental allergic dermatitis, and food allergic dermatitis.

Also disclosed are computer systems. In some embodiments, a computer system is configured to indicate dermatologic and/or respiratory disease states in cats, and includes one or more processors and one or more computer-readable hardware storage devices that store instructions executable by the one or more processors. The instructions may configure the computer system to receive sequenced microbial DNA data from an oral swab sample taken from a cat; map the sequenced microbial DNA to identify which specific one or more microbial species are present in the oral sample, wherein identifying the specific one or more microbial species results in generation of an oral microbial profile for the cat; calculate relative abundance of different microbial species to further build the oral microbial profile; compare the oral microbial profile against a database of defined microbial profiles, wherein the database identifies correlations between (i) profiles that include one or more microbial species and their relative abundance, and (ii) corresponding dermatologic and/or respiratory diseases; and based on a result of comparing the oral microbial profile against the database of defined microbial profiles, generate a risk score indicating a likelihood that the cat has a specific dermatologic and/or respiratory disease. In response to generating the risk score, the instructions may further configure the computer system to generate a report outlining and/or presenting the risk score and prescribing a therapeutic treatment and/or an at-home treatment protocol suitable for addressing (e.g., treating, arresting, and/or preventing) the specific dermatologic and/or respiratory disease. The therapeutic treatment protocol may be influenced by the stage or severity of the dermatologic and/or respiratory disease state, which is indicated by or correlated to the risk score. The dermatologic and/or respiratory disease states are selected from the group consisting of asthma, atopic dermatitis, flea allergic dermatitis, environmental allergic dermatitis, and food allergic dermatitis.

For example, in some embodiments, the risk score may incorporate or correlate to approximately three (3) risk assessment categories based on the risk/probability score generated: a 0.0-0.33 bracket is classified as ‘low risk’ of having a dermatologic condition; >0.33-0.66 is classified as ‘medium risk’ for having a dermatologic condition; and >0.66-1.0 is classified as ‘high risk’ for having a dermatologic condition. For example, a risk score of 0.34 would meet the threshold for categorizing a cat as being at medium risk for having a dermatologic condition. The granularity of the risk score and/or the number of categories may change as more data is added to the systems and methods.

In some embodiments, the therapeutic treatment or at-home care protocol can alter the composition of the oral microbiome of the cat directly or as a byproduct of the treatment of a specific dermatologic and/or respiratory disease. In some embodiments, altering the composition of the cat's oral microbiome treats and/or addresses the specific dermatologic and/or respiratory disease. In some embodiments, the therapeutic treatment repairs the cat's oral microbiome. In some embodiments, repairing the cat's oral microbiome brings the cat's oral microbiome more in line with the oral microbiome (or defined oral microbial profile) of a healthy cat-both in terms of the specific one or more microbial species present and their relative abundance. In some embodiments, the therapeutic treatment or at-home care protocol is designed to maintain the composition of the oral microbiome of the cat. In some embodiments, the therapeutic treatment protocol is designed to stimulate a metabolic output of the cat's oral microbiome. Stimulating a metabolic output of the cat's oral microbiome may include using known enzymatic pathway analysis tools to provide an additional dimension to the existing microbial composition data to further characterize disease signatures and improve predictive disease models.

Illustrative embodiments and non-limiting examples of the present disclosure include:
Example 1. A method for screening for, detecting, and/or preventing dermatologic and/or respiratory disease in domestic cats, the method comprising:
   obtaining an oral microbial profile for a cat, the oral microbial profile comprising one or more microbial species present in an oral sample from the cat and a quantity or abundance of the one or more microbial species in the oral sample;
   comparing the oral microbial profile to information in a database that identifies weighted correlations between:
   (i) occurrence and/or prevalence of one or more dermatologic and/or respiratory diseases in cats; and
   (ii) presence and/or abundance of various microbial species in oral microbiomes of cats, wherein the various microbial species comprise the one or more microbial species in the oral sample;
   generating a risk score indicating a likelihood that the cat has the one or more dermatologic and/or respiratory diseases based on one or more matches between the oral microbial profile of the cat and the information in the database; and
   categorizing the cat as developing the one or more dermatologic and/or respiratory diseases when the risk score meets or exceeds a predetermined threshold and, optionally, prescribing a therapeutic treatment protocol suitable for treating, mitigating, or preventing the development, advancement, or recurrence of the one or more dermatologic and/or respiratory diseases when the risk score meets or exceeds a predetermined threshold.
Example 2. The method of Example 1 further comprising administering the therapeutic treatment protocol to the cat or confirming that the therapeutic treatment protocol has been administered to the cat, wherein the therapeutic treatment protocol is sufficient to alter the oral microbial profile of the cat.
Example 3. The method of Example 1, wherein obtaining the oral microbial profile for the cat comprises:
   obtaining nucleic acid sequence data corresponding to microbial nucleic acid obtained from the oral sample;
   analyzing the nucleic acid sequence data to identify the one or more microbial species present in the oral sample and, optionally, quantifying the one or more microbial species; and
   generating the oral microbial profile for the cat based on the identified and, optionally, quantified one or more microbial species.
Example 4. The method of Example 3, wherein obtaining the microbial nucleic acid sequence data comprises:
   sequencing microbial nucleic acid from the oral sample; and, optionally,
   isolating the microbial nucleic acid from the oral sample.
Example 5. The method of Example 4, wherein isolating the microbial nucleic acid from the oral sample comprises:
   performing heat treatment on the oral sample; and
   performing magnetic SPRI beads-based nucleic acid extraction on the heat-treated oral sample, with or without the addition of protein digesting reagents and detergents, to extract the microbial nucleic acid from the oral sample.
Example 6. The method of Example 3, wherein analyzing the microbial nucleic acid sequence data comprises one or more of:
   demultiplexing the nucleic acid sequence data;
   trimming the nucleic acid sequence data;
   mapping one or more unmapped reads onto a reference genome of the cat and/or onto existing microbial reference genomes;
   classifying one or more reads as feline from the nucleic acid sequence data after mapping;
   classifying one or more reads as microbial from the nucleic acid sequence data after mapping;
   quantifying the one or more microbial reads;
   transforming the quantified one or more microbial reads to account for sequence coverage biases using methods such as pairwise log ratio transformation; and
   comparing compositional abundance patterns in the transformed one or more microbial reads against compositional abundance patterns in transformed data in a reference database comprising samples from cats that do not suffer from dermatologic and/or respiratory diseases, as well as samples from cats that suffer from specific dermatologic and/or respiratory diseases.
Example 7. The method of Example 1, wherein comparing the oral microbial profile for the cat to the information in the database comprises one or more of:
   calculating the abundance of the one or more microbial species in the oral sample;
   identifying the one or more microbial species in the oral sample; and
   comparing the abundance of the identified one or more microbial species in the oral sample to the presence and/or abundance of various microbial species in the oral microbiome of cats contained in the database.
Example 8. The method of Example 1, wherein generating the risk score comprises one or more of:
   identifying one or more similarities between compositional abundance(s) of the one or more microbial species in the oral sample and compositional abundance(s) of various microbial species in the oral microbiomes of cats contained in the database;
   identifying one or more matches between the identities of the one or more microbial species in the oral sample and the presence of various microbial species in the oral microbiome of cats contained in the database;
   quantifying the identified one or more similarities between the compositional abundance(s) of the one or more microbial species in the oral sample and the compositional abundance(s) of the one or more microbial species in the oral microbiomes of cats contained in the database; and
   identifying a presence of one or more predictive microbial species in the oral sample.
Example 9. The method of Example 1, wherein the one or more dermatologic and/or respiratory diseases are selected from the group consisting of asthma, atopic dermatitis, food allergic dermatitis, flea allergic dermatitis and environmental allergic dermatitis.
Example 10. The method of Example 1 further comprising:
   generating a report presenting (i) the risk score, (ii) an indication of developing the one or more dermatologic and/or respiratory diseases when the risk score meets or exceeds the predetermined threshold, (iii) a timing recommendation, (iv) optionally, one or more at home practices to improve dermatologic and/or respiratory health, (v) optionally, one or more diagnostic steps to diagnose the one or more dermatologic and/or respiratory diseases when the risk score meets or exceeds the predetermined threshold, and (vi) optionally, a prescription for the therapeutic treatment protocol; and, optionally,
   communicating the generated report electronically to an owner of the cat and/or their veterinarian.
Example 11. The method of Example 1, wherein the therapeutic treatment protocol is sufficient to alter the oral microbial profile of the cat.
Example 12. A computer system configured to indicate or predict dermatologic and/or respiratory disease in cats, the computer system comprising:
   one or more processors; and
   one or more computer-readable hardware storage devices having stored thereon instructions that are executable by the one or more processors to configure the computer system to:
   receive microbial nucleic acid sequence data corresponding to microbial nucleic acid obtained from an oral sample taken from a cat;
   analyze the microbial nucleic acid sequence data to identify one or more microbial species present in the oral sample and quantify the one or more microbial species;
   generate an oral microbial profile for the cat based on the identified one or more microbial species and their respective abundances;
   compare the oral microbial profile to information in a database that identifies weighted correlations between:
   (i) occurrence and/or prevalence of one or more dermatologic and/or respiratory diseases in cats; and
   (ii) presence and/or abundance of various microbial species in oral microbiomes of cats, wherein the various microbial species comprise the one or more microbial species in the oral sample;
   identify one or more matches between the oral microbial profile and the information in the database;
   generate a risk score indicating a likelihood that the cat has the one or more dermatologic and/or respiratory diseases based on the one or more matches between the oral microbial profile and the information in the database; and, optionally,
   diagnose the cat as “developing” the one or more dermatologic and/or respiratory diseases when the risk score meets or exceeds a predetermined threshold,
   prescribe a therapeutic treatment protocol suitable for treating or preventing the one or more dermatologic and/or respiratory diseases when the risk score meets or exceeds the predetermined threshold,
   generate a report indicating (i) the risk score, (ii) an indication of developing the one or more dermatologic and/or respiratory diseases when the risk score meets or exceeds the predetermined threshold, (iii) a timing recommendation, (iv) optionally, one or more at home practices to improve dermatologic and/or respiratory health, (v) optionally, one or more diagnostic steps to diagnose the one or more dermatologic and/or respiratory diseases when the risk score meets or exceeds the predetermined threshold, and (vi) a prescription for the therapeutic treatment protocol, and/or
   communicate the generated report electronically to an owner of the cat and/or their veterinarian.
Example 13. The computer system of Example 12, wherein the instructions further configure the computer system to analyze metagenomic sequence data from the oral sample and map one or more unmapped sequence reads to a feline reference genome and/or map one or more sequence reads to microbial reference genomes and, optionally, classify the reads as microbial or feline.
Example 14. The computer system of Example 13, wherein the instructions further configure the computer system to identify at least one unmapped sequence read of the metagenomic sequence data and, optionally, classify the at least one unmapped read.
Example 15. The computer system of Example 13, wherein feline oral microbiome samples having fewer than 10,000 classified microbial reads or more than 500,000 classified microbial reads are excluded from the comparison of the oral microbial profile for the cat against a database of defined microbial profiles.
Example 16. The computer system of Example 12, wherein the instructions further configure the computer system to calculate an abundance of the one or more microbial species present in the oral sample.
Example 17. The computer system of Example 16, wherein the abundance of the specific one or more microbial species present in the oral sample correlates to whether the specific one or more microbial species is a predictive microbial species for the specific dermatologic and/or respiratory disease.
Example 18. The computer system of Example 16, wherein the instructions further configure the computer system to perform a pairwise log ratio comparison of the microbial abundance of the oral sample against the information in the database.
Example 19. The system of Example 18, wherein the specific one or more microbial species is a predictive microbial species when 50% or more of the maximum possible pairwise log ratio comparisons involving this microbe are significantly different when compared between a disease and a control cohort.
Example 20. A method for predicting the development of a dermatologic and/or respiratory disease in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more dermatologic and/or respiratory diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more dermatologic and/or respiratory diseases based on one or more matches between the oral microbial profile and the information in the database; and
   indicating the cat as developing the one or more dermatologic and/or respiratory diseases when the risk score meets or exceeds a predetermined threshold.
Example 21. A method for diagnosing a dermatologic and/or respiratory disease in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more dermatologic and/or respiratory diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more dermatologic and/or respiratory diseases based on one or more matches between the oral microbial profile and the information in the database; and
   diagnosing the cat as developing the one or more dermatologic and/or respiratory diseases when the risk score meets or exceeds a predetermined threshold.
Example 22. A method for treating a dermatologic or respiratory disease in a cat, the method comprising:
   obtaining an oral sample from a cat, the oral sample comprising one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify the one or more microbial species present in the oral sample and, optionally, quantifying the one or more microbial species;
   generating an oral microbial profile for the cat based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more dermatologic or respiratory diseases in cats; and
   (ii) presence and/or abundance of various microbial species in oral microbiomes of cats, wherein the various microbial species comprise the one or more microbial species in the oral sample;
   generating a risk score indicating a likelihood of the cat developing the one or more dermatologic or respiratory diseases based on one or more matches between the oral microbial profile and the information in the database;
   diagnosing the cat as developing the one or more dermatologic or respiratory diseases when the risk score meets or exceeds a predetermined threshold; and
   administering a therapeutic treatment, wherein the therapeutic treatment is sufficient to treat the one or more dermatologic or respiratory diseases.
Example 23. The method of Example 22, wherein the dermatologic or respiratory disease is selected from the group consisting of asthma, atopic dermatitis, food allergic dermatitis, flea allergic dermatitis and environmental allergic dermatitis.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Figures, like reference numerals may be utilized to designate corresponding or similar parts in the various Figures, and the various elements depicted are not necessarily drawn to scale, wherein:

FIGS. 1A-1B illustrate a dermatologic health test workflow and oral microbiome reference database construction.

FIGS. 2A-2D illustrate a distribution of the average log ratio difference scores between pairwise microbial interactions associated with healthy cohorts and (A) atopic dermatitis, (B) food allergic dermatitis, (C) flea allergic dermatitis, (D) environmental allergic dermatitis.

FIGS. 3A-3D illustrate sensitivity and specificity of the feline dermatologic health test based on a 2-component Gaussian mixture model. Sensitivity refers to the ability of the disclosed embodiments to detect cats known to suffer from a dermatologic condition. Specificity refers to the ability of the disclosed embodiments to detect cats in the healthy control cohorts as not suffering from a dermatologic condition.

FIG. 4 illustrates overlap of oral microbiome predictive microbes characteristic of feline atopic dermatitis, food allergic dermatitis, flea allergic dermatitis or environmental allergic dermatitis.

FIG. 5 illustrates microbial species richness as a function of number of sequencing reads, comparing data from two different types of metagenomic whole genome sequencing (WGS) library preparations-a ligation-based approach versus a tagmentation-based approach (such as the Illumina Nextera DNA Flex Library Preparation Kit).

FIG. 6 illustrates a distribution of the average log ratio difference scores between pairwise microbial interactions associated with healthy cohorts and asthma cohorts.

FIG. 7 illustrates sensitivity and specificity of the feline health test for asthma and healthy cohorts based on a 2-component Gaussian mixture model.

### DETAILED DESCRIPTION

Variations in the microbial composition of the mouth (i.e., the oral microbiome) may have associations with certain dental and systemic diseases. This research area is still young and studies on human subjects demonstrating these associations in a comprehensive manner have only been published in the last decade or less. Studies on this topic in companion animals, such as cats and dogs, have been limited. Nutritional and environmental factors, as well as present disease states, may play an important role in the dynamic microbial composition of a cat's mouth (i.e., their oral microbiome). With the mouth being the first line of defense from a constant exposure to foreign microbes, the oral microbiome has evolved to be competitive and territorial. It is comprised of microbes that excel at defending their territory and typically avoid being replaced by foreign invaders, including pathogens.

However, dysbiosis inducing events such as poor diet, poor dental hygiene, the onset of systemic diseases or environmental changes, can lead to pathogenic microbes colonizing disproportionately large parts of the oral cavity (and, thus, altering the oral microbiome). This colonization can be associated with pathological disease states. Understanding the composition of the oral microbiome can provide information not only about the health of the oral tissues, but also about the general health of the animal or human. For example, oral microbiome characteristics have been linked with diseases such as inflammatory bowel disease (IBD), various cancers, and atopic dermatitis, among others. Oral microbiome characteristics may also be linked with asthma, another inflammatory and allergenic condition. The information provided by the state of the oral microbiome may also be used to manage the health and wellbeing of a pet.

The field of oral microbiome research in companion animals has received little focus and it is still in its infancy. Existing studies base their conclusions on small sample sizes and outdated culture-based techniques for querying the microbiome. It is estimated that only around 2% of all existing bacteria can be cultured in the laboratory. This means that studies relying on this method for microbial classification may miss many important microbial organisms. Additionally, false emphasis might be placed on particular species, simply because they could be cultured and measured. This problem is compounded by the fact that lab culturing provides a very bacteria-centric view of the microbiome, often ignoring other microorganisms such as fungi, protozoa, archaea and viruses.

Interrogating the oral microbiome of a cat can be accomplished by using an oral (saliva) sample. Saliva sampling kits have gained popularity in recent years as tests for ancestry and microbial infection have become more prevalent. Available direct-to-consumer microbiome tests typically rely on a technique called ‘16S rRNA gene sequencing,’ which utilizes Next Generation Sequencing (NGS). While this technique provides substantially more information than early bacterial culturing efforts, it can only be used for identifying bacterial species (and some archaea) present in the microbiome. In most cases, these tests do not provide sufficient resolution to reliably, and consistently, identify bacteria beyond the genus level of taxonomic classification. Therefore, in most cases, the test results do not provide the exact species or strain of bacteria comprising the microbiome. Thus, data-driven conclusions using these results are vague and rely on approximation. Moreover, it is well-known that the microbiomes of different sites of the body can be composed of viruses, protozoa, and fungal species, in addition to bacteria and archaea. This means that the 16S rRNA gene sequencing approach zooms in on just one part of the microbiome, ignoring the rest.

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments and is not necessarily intended to limit the scope of the present disclosure and/or the claimed invention.

Presently disclosed are computer systems, systems and methods for the identification, screening, indication, diagnosis, and/or treatment of dermatologic and/or respiratory disease states in cats. Embodiments of the disclosed subject matter describe a method for interrogating the oral microbiome of a domestic cat for the purpose of detecting microbe compositional abundance trends associated with dermatologic and/or respiratory diseases in cats. Detecting, identifying and/or quantifying microbe compositional abundance trends enables a practitioner to screen for and/or indicate whether a cat has a particular dermatologic and/or respiratory disease state. Detecting and identifying dermatologic and/or respiratory disease states enables the practitioner and pet owner to treat and/or delay the disease progression, and in some cases even potentially prevent the future recurrence of the dermatologic and/or respiratory disease state.

Disclosed methods may compare, for example, a cat's oral microbiome to the oral microbiomes of cats reported by their owners and/or a veterinary professional to have been diagnosed with asthma, atopic dermatitis, food allergic dermatitis, flea allergic dermatitis, or environmental allergic dermatitis. The comparison is carried out using a reference database containing defined microbial profiles, associating one or more microbial species and their respective compositional abundance(s) with one or more dermatologic conditions.

Disclosed systems and methods can comprise a painless oral swab sample collection. Accordingly, the oral microbiome can be surveyed via buccal, supragingival or subgingival sampling. Such sampling does not require anesthetizing the animal and can be performed by the pet owner at their home or by the veterinarian at the clinic. The disclosed systems and methods can potentially serve as an early indicator of dermatologic disease-associated processes not yet formally diagnosed. Routine use may enable identification of early-stage dermatologic diseases, driving more cats to the veterinary office early on and reducing animal suffering. Earlier identification of dermatologic disease states beneficially saves costs and improves the quality of life of cats. Earlier identification of dermatologic and respiratory disease states also means more treatment options are available when the dermatologic and respiratory disease is diagnosed and identified.

### Defined Microbial Profiles Contained in the Reference Database

With the mouth being the first line of defense from constant exposure to foreign microbes, the oral microbiome has evolved to be competitive and territorial. It is comprised of microbes that excel at defending their territory and are typically able to resist being replaced by foreign invaders, including pathogens. These microbes are generally present when a cat is healthy and would represent a healthy microbial profile of the oral microbiome. When the cat is suffering from a dermatologic or respiratory condition, the composition of the oral microbiome may be altered by the presence of foreign or pathogenic microbial species and/or altered abundance ratios of and between different microbes. Such an alteration in the composition of the oral microbiome might be represented by a pathogenic profile. In some cases, the presence of particular foreign and/or pathogenic microbial species, and their abundance relative to other microbes in the oral cavity, is correlated with the cat suffering from a particular dermatologic and/or respiratory condition.

Identification of the particular (one or more) microbial species (and their respective relative abundance(s)) correlated with particular dermatologic and/or respiratory disease states enables pre-diagnostic screening for the dermatologic and/or respiratory disease state in a cat exhibiting the presence of the identified (one or more) microbial species. In other words, identification and/or indication of the dermatologic and/or respiratory disease state may be correlated to the cat exhibiting a particular pathogenic profile.

The gold standard for the comprehensive study of the microbiome is shotgun metagenomic sequencing, which allows capturing complete or near-complete genomes of organisms across all domains of life, not just bacteria and archaea. The gold standard for metagenomic DNA extraction includes a process called bead-beating. It is recommended for complete microbial cell lysis when studying the abundance and composition of the microbiome. The process helps break apart thicker cell walls, such as those of gram-positive bacteria. It is achieved by rapidly agitating samples with grinding media (balls or beads) in a bead beater.

In some exemplary embodiments, the disclosed systems and methods do not use bead-beating for metagenomic DNA extraction and purposefully abandon such a process. The reason for this is that bead-beating can also introduce significant DNA degradation that interferes with downstream sample processing and can therefore lower the quality of the generated metagenomic sequencing library. Since the disclosed systems and methods, according to one embodiment, do not use bead-beating, it is likely that the oral microbiome data in the resulting analyses suffer from under-representation of gram-positive bacteria. Nonetheless, it allows the recognition of disease-characteristic patterns. The disclosed systems and methods also enable microbial identification and classification down to the species or, in some instances, the strain level, unlike 16S rRNA gene sequencing.

Some results from research of the human oral microbiome suggest that different allergy-related conditions can be characterized by similar changes in the oral microbiome. This view is paralleled in cats due to the fact that some overlap in microbial species important for each of the feline dermatologic conditions of interest (atopic dermatitis, food allergic dermatitis, flea allergic dermatitis, environmental allergic dermatitis) is observed. However, also identified were microbes whose compositional abundance in the oral microbiome are predictive specifically of atopic dermatitis, food allergic dermatitis, flea allergic dermatitis or environmental allergic dermatitis. This suggests that there are microbial profiles associated with specific dermatitis pathologies, in addition to the existence of a core set of microbes associated with atopic and allergic dermatitis-related diseases in general. This also suggests there may be microbial profiles associated with specific respiratory pathologies, such as asthma.

Using shotgun metagenomic oral microbiome sequencing of 38,000 domestic cats and compositional data analysis techniques, a comprehensive survey of the feline oral microbiome was executed, identifying 8,344 microbial species present in the feline oral microbiome. Whether a domestic cat included in the shotgun metagenomic sequencing suffered from a particular dermatologic or respiratory condition was determined by asking their owner through a survey if the cat had been formally diagnosed by a veterinarian as suffering from a particular dermatologic or respiratory condition (e.g., asthma, atopic dermatitis, food allergic dermatitis, flea allergic dermatitis, environmental allergic dermatitis, etc.).

The reference database is a weighted correlation database and contains at least the identified 8,344 microbial species present in the feline oral microbiome. On average, 606 microbial species per cat were identified, 97% of which were classified as bacteria and archaea, 0.27% as DNA viruses (RNA viruses cannot be detected with shotgun metagenomic sequencing), 0.02% as phages and <2% as fungi. The various microbial species identified as being involved in and contributing to a specific dermatologic or respiratory disease are compiled into a “defined microbial profile.” The defined microbial profile is a list or collection of identified one or more microbial species, and their respective relative abundances, known to contribute to and/or be involved in a specific dermatologic or respiratory disease condition. In some embodiments, defined microbial profiles may include percentages of gram-positive microbes and ratios of gram-positive microbes to gram negative microbes, in addition to the identities (i.e., genus and species) of microbes. In some embodiments, defined microbial profiles may indicate the relative abundance (increased or decreased) of the one or more microbial species. (See Tables 1-16 below).

For example, a defined microbial profile may include a set of seventy (70) microbes that are predictive for four dermatologic conditions (atopic dermatitis, food allergic dermatitis, flea allergic dermatitis, or environmental allergic dermatitis), as well as microbes specifically predictive for one of the four dermatologic conditions (atopic dermatitis, food allergic dermatitis, flea allergic dermatitis, environmental allergic dermatitis). A defined microbial profile may also be compiled for respiratory conditions, such as asthma. “Predictive microbes” are discussed more fully below. The defined microbial profile may rank and/or weigh each included microbial species by how frequently and in what proportions a certain microbe is observed in animals suffering from the specific dermatologic or respiratory condition, as deduced by consulting a reference database. How much any one microbial species contributes to a specific dermatologic and/or respiratory disease condition is correlated to how often a microbial species shows up (or is present) in the oral microbiome while an animal is suffering from a specific dermatologic and/or respiratory disease condition. How much any one microbial species contributes to a specific dermatologic and/or respiratory disease condition also correlates to how consistently such microbial species demonstrates significantly different relative abundances from other oral microbes when compared to healthy control samples.

The defined microbial profiles contained in the reference database also include defined microbial profiles of healthy cats that are not suffering from a dermatologic and/or respiratory condition. For example, the defined microbial profile of healthy cats lists and identifies the microbial species present in the oral microbiome, as well as their relative abundances, when no dermatologic and/or respiratory condition is present. A healthy defined microbial profile may establish a baseline or control for the microbial species present and their relative abundance. Any deviations from this profile may enable a practitioner to predict and/or indicate, for example, a cat's likelihood of suffering from a dermatologic and/or respiratory condition. Similarly, deviations from the healthy defined microbial profile may enable a practitioner in diagnosing a cat as suffering from a dermatologic and/or respiratory condition prior to the onset of symptoms for that dermatologic and/or respiratory condition.

The defined microbial profile for each dermatologic and/or respiratory disease state is compared to the defined microbial profile for a healthy cat to determine any differences between the dermatologic and/or respiratory disease states and a healthy state. In some embodiments, the comparisons are pairwise log ratio comparisons. For example, there may be some overlap in the oral microbiome of a healthy cat and a cat suffering from atopic dermatitis. A comparison of the healthy defined microbial profile to the atopic dermatitis defined microbial profile would identify common microbial species seen in similar abundances between the two. Any microbial species not common between the two microbial profiles, or any microbial species seen in significantly different proportions between the two profiles, would confirm the involvement of that microbial species in the development of atopic dermatitis. Identification of such a microbial species in a cat's oral microbiome would be indicative of the cat having atopic dermatitis.

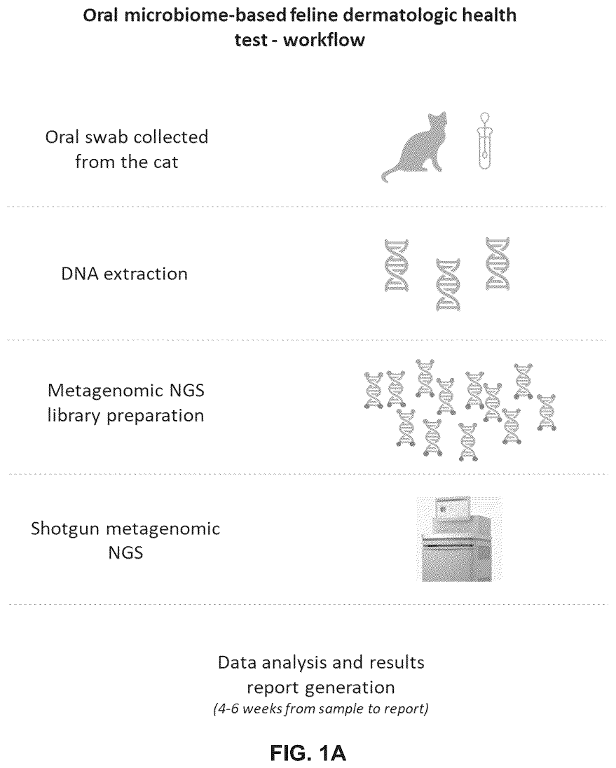

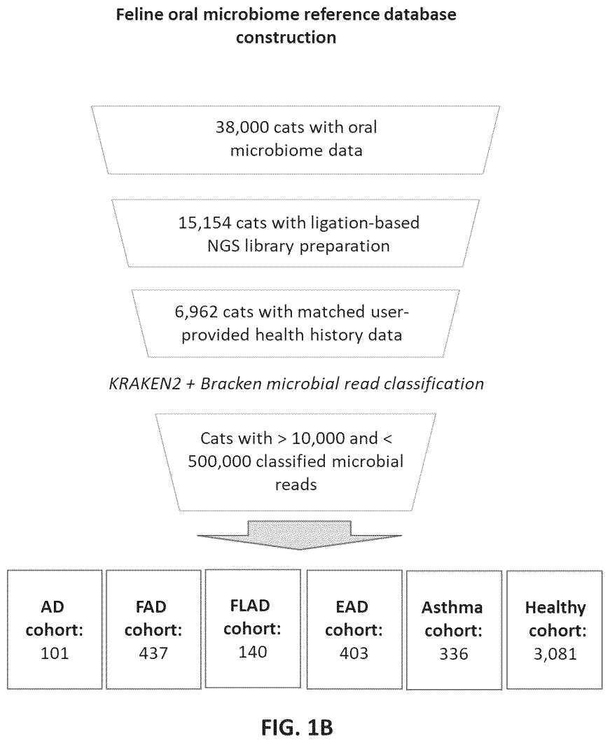

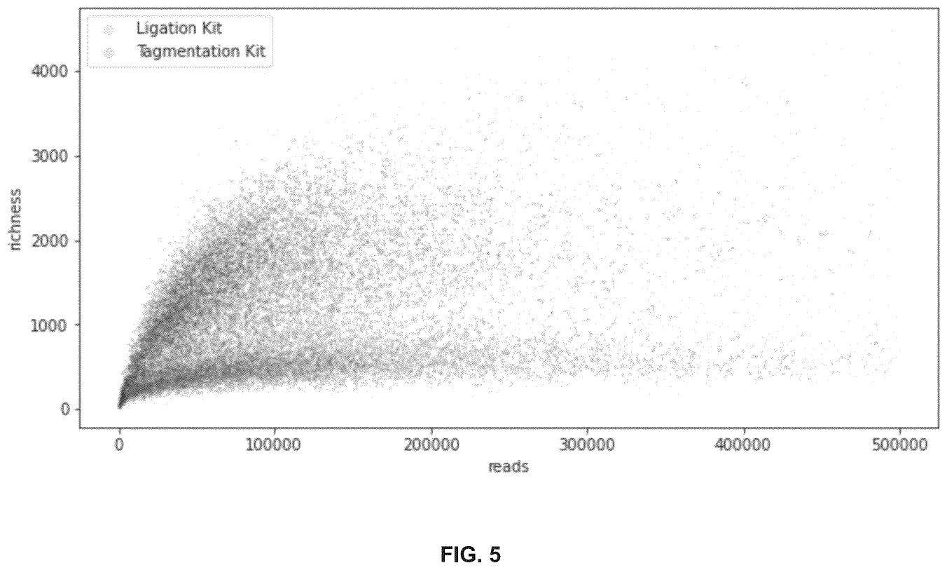

FIGS. 1A-1B illustrate a dermatologic health test workflow and construction of the oral microbiome reference database using feline subjects. In FIG. 1A, the feline dermatologic health test workflow includes collecting an oral swab from the cat in a DNA preservation solution, extracting and preparing the DNA for shotgun metagenomic next generation sequencing (NGS), sequencing the DNA, data analysis and the generation of a report presenting risk assessment for different dermatologic diseases based on the state of the oral microbiome. The report may be accompanied by treatment recommendations tailored to the results. In FIG. 1B, the feline oral microbiome reference database was constructed through applying sequential filters on the initial database of 38,000 cats. First, all data from tagmentation-based NGS library preparation samples was removed. This was done due to an observed effect of the library preparation method on microbial species richness (see FIG. 5). The ligation-based method was preferred because the number of sequencing reads per sample had minimal impact on the number of microbial species detected. In addition, Tn5 transposase assisted tagmentation is known to introduce GC sequencing bias, particularly in metagenomic communities. However, tagmentation-based NGS library preparation may be included in some embodiments.

Next, samples lacking an accompanying phenotype/health history record for the cat were excluded. The microbial sequence data from the sample is identified, classified and mapped. After classification of the microbial reads in each sample using KRAKEN2 and Bracken, samples with fewer than 10,000 and more than 500,000 classified microbial reads were removed. The remaining cats/samples were placed into cohorts. This resulted in an atopic dermatitis cohort (AD; n=101), food allergic dermatitis (FAD; n=437), flea allergic dermatitis (FLAD; n=140), and environmental allergic dermatitis (EAD; n=403).

Though FIGS. 1A-1B illustrate a dermatologic health test workflow and construction or the oral microbiome reference database, it is to be understood that the same health test workflow was performed for respiratory conditions (e.g., asthma). Thus, an asthmatic cohort (n=336) was obtained, classified, mapped, and, likewise, added to the oral microbiome reference database. Use of the oral microbiome reference database in conjunction with the disclosed computer systems, systems and methods enables a practitioner to screen for, indicate, identify, diagnose, and/or treat disease states in cats. The disease states include, at least, AD, FAD, FLAD, EAD, and asthma.

### Identifying Predictive Microbes

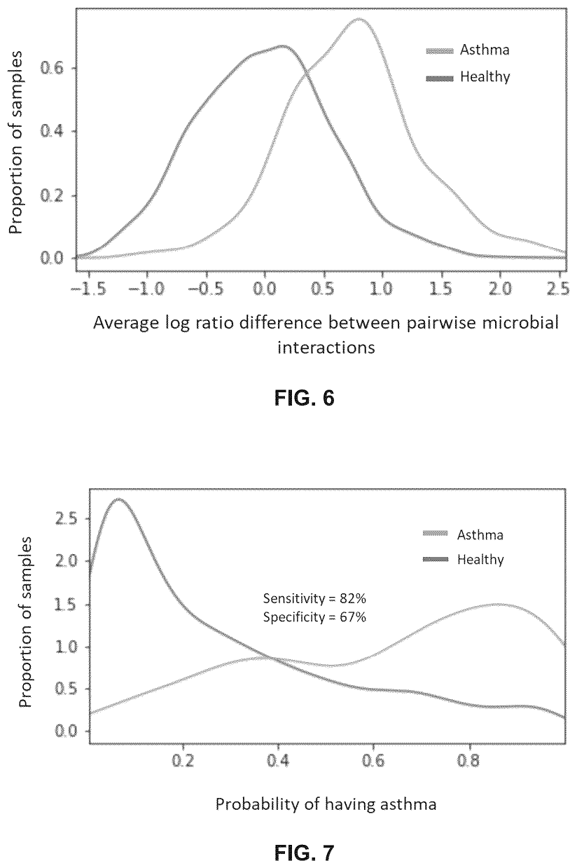

As a first step towards identifying microbes significantly correlated with each dermatologic and/or respiratory condition, Pairwise Log-Ratio (PLR) transformation was performed on the Bracken output species level read counts. Next, the significant PLR comparisons (p-value <0.01) were identified between the control and a condition by performing a z-test. The healthy cohort was compared to the AD, FAD, FLAD and EAD cohorts. A healthy cohort was also compared to an asthmatic cohort. (See FIGS. 6-7).

The frequency of each microbial species in all significant PLRs was assessed. Only microbial species where 50% or more of their maximum possible comparisons with other species were significant were kept. This measure was used as a proxy for the importance of different microbial species in the four dermatologic and one respiratory conditions of interest. These microbial species are “predictive microbial species” for each dermatologic and respiratory condition.

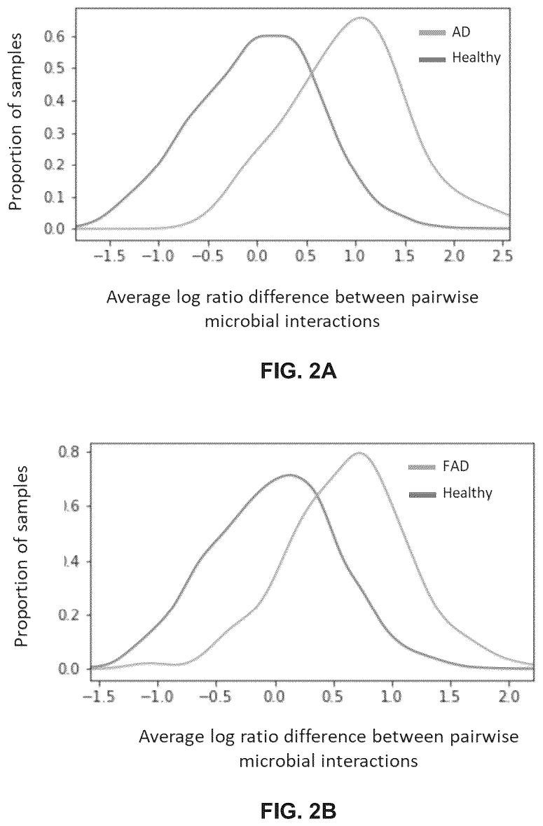

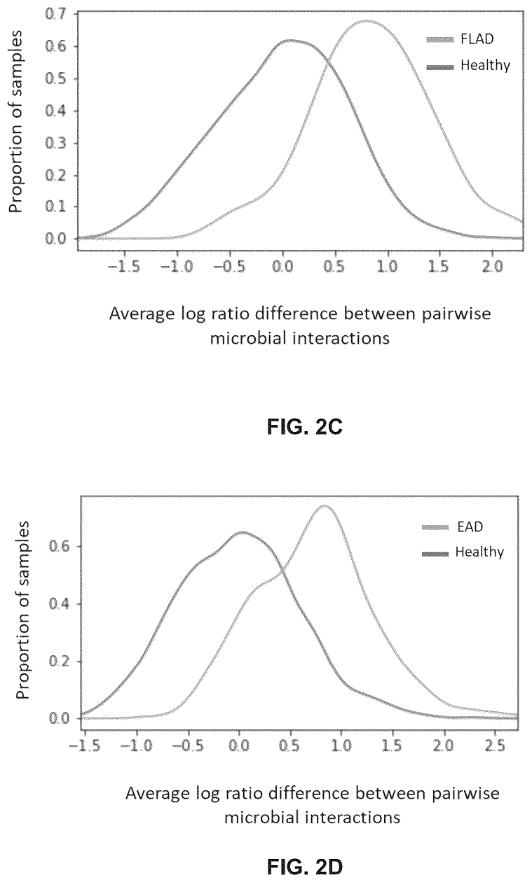

In order to identify population-wide microbial compositional abundance patterns characteristic of atopic dermatitis, food allergic dermatitis, flea allergic, and environmental allergic dermatitis, each sample was scored by comparing the predictive pairwise log-ratios (pPLRs) of the sample to the mean pPLRs of controls, taking into account the direction and magnitude of the difference. FIGS. 2A-2D illustrate a distribution of the average log ratio difference scores between pairwise microbial interactions associated with healthy cohorts and atopic dermatitis, food allergic dermatitis, flea allergic, environmental allergic dermatitis. FIG. 6 illustrates a distribution of the average log ratio difference score between pairwise microbial interactions associated with healthy cohorts and asthmatic cohorts.

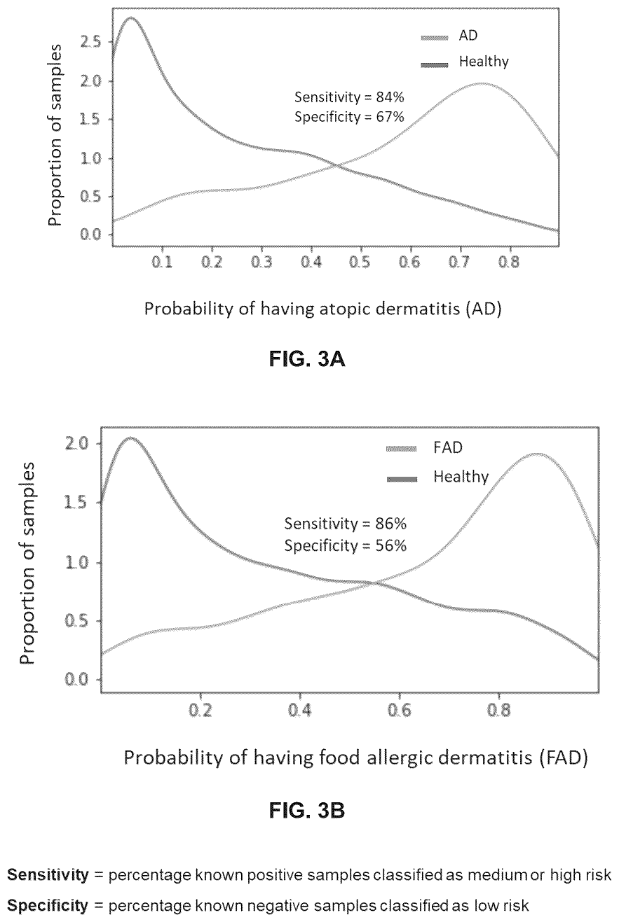

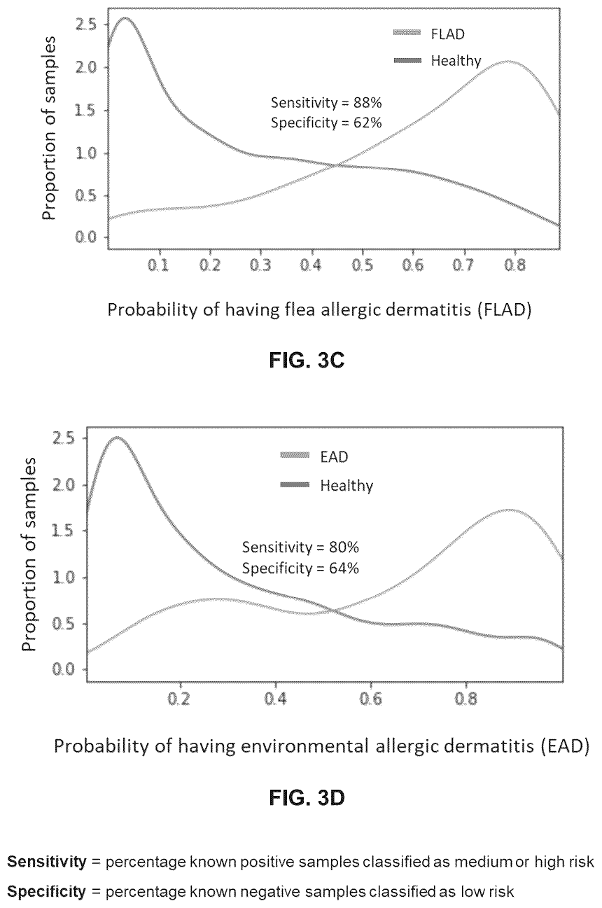

Next, we fitted four (4) Gaussian mixture models (one for each dermatologic condition) with two (2) components each—healthy cohort and dermatologic condition-onto the distribution of the average log ratio difference score between pairwise microbial interactions. This modeling approach generates a 0 to 1 score for each sample, which represents the probability that the sample belongs to the control cohort or to the respective dermatologic condition cohort. FIGS. 3A-3D plot the probability that samples belonging to four of the dermatologic disease cohorts (atopic dermatitis, food allergic dermatitis, flea allergic, environmental allergic dermatitis) and the control samples would be classified as belonging to their respective cohorts based on each sample's compositional abundance of predictive microbes. A bimodal probability distribution consistent with sample identity was observed between the dermatologic condition and control in all cases. There was a minority of disease samples forming a small peak closer to 0 and a small set of control samples forming a slight peak closer to 1. FIG. 7 plots the probability that samples belonging to the asthmatic cohort and the control samples would be classified as belonging to their respective cohorts based on each sample's compositional abundance of predictive microbes.

The defined microbial profile for each dermatologic and/or respiratory disease state (asthma, atopic dermatitis, food allergic dermatitis, flea allergic, and environmental allergic dermatitis) is compared to the defined microbial profile for a healthy cat to determine and quantify differences and commonalities in microbial species and their abundance between the dermatologic disease states and a healthy state. The defined microbial profiles for each dermatologic disease state are also compared to each other to identify overlapping microbial species common to each dermatologic disease state. The defined microbial profile for asthma underwent similar comparisons to determine and quantify differences and commonalities in microbial species and their abundance between asthma and a healthy state, as well as to identify overlapping microbial species common to each disease state.

The defined microbial profiles for each dermatologic or respiratory disease state and a healthy control state undergo a pairwise log ratio (PLR) transformation. The PLR transformation corrects for potential sequencing coverage differences between samples by scaling microbial abundances relative to each microbe instead of a constant scaling factor. Next, a z-test between PLRs from each disease state versus the control state is performed. A p-value of approximately <0.01 serves as a threshold value for significant PLR comparisons. For each microbial species identified in a defined microbial profile for a dermatologic or respiratory disease state, the number of significant PLR comparisons (as defined by the p-value) that microbial species shows up in is counted. If the number of significant PLR comparisons is at least 50% of all possible PLR comparisons for that microbe, the microbial species is deemed a “predictive microbe.” This process may be repeated for each dermatologic or respiratory disease state of interest. In other words, through z-test identification of significant PLR comparisons, predictive microbes can be identified for asthma, atopic dermatitis, food allergic dermatitis, flea allergic and/or environmental allergic dermatitis. Table 1 provides examples of identified predictive microbes for atopic dermatitis, food allergic dermatitis, flea allergic and environmental allergic dermatitis. Table 2 provides examples of identified predictive microbes for asthma.

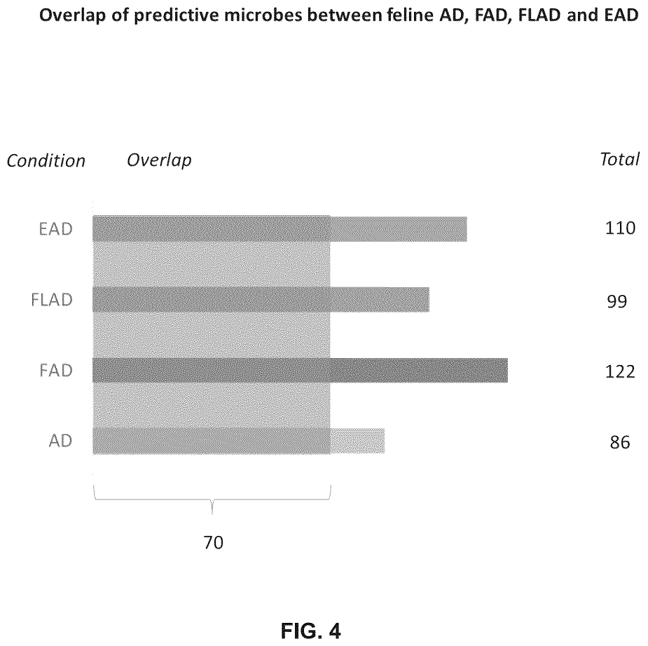

As outlined in Table 1, 86 predictive microbes for atopic dermatitis, 122 for food allergic dermatitis, 99 for flea allergic dermatitis, and 110 for environmental allergic dermatitis were identified. The predictive microbes for each dermatologic disease were identified based on PLR microbial abundance comparisons between healthy/control defined microbial profiles and the defined microbial profiles of cats suffering from one of four dermatologic conditions (Sec FIG. 4). Seventy (70) microbes were identified as predictive for the four dermatologic conditions (atopic dermatitis, food allergic dermatitis, flea allergic and environmental allergic dermatitis), though each condition has its own specific set of predictive microbes, differentiating it from other conditions. Plotting the average log ratio difference between significant pairwise microbial interactions in a dermatologic or respiratory condition versus control samples allowed separation of sample populations based on their dermatologic or respiratory disease status. (Sec FIGS. 2A-2D and 6). However, some overlap between the populations was observed, meaning that for a certain set of samples, their compositional abundance of predictive microbes could be interpreted as either consistent with the control population or the respective dermatologic disease population.

Tables 3-7 outline the percentages of microbes identified or associated with the dermatologic or respiratory disease states of interest (e.g., asthma, atopic dermatitis, food allergic dermatitis, flea allergic, and environmental allergic dermatitis). Tables 8-12 outline the relative increased or decreased abundance for each predictive microbe for each dermatologic and/or respiratory disease state of interest. This data (regarding relative abundances, percentages, and ratios of gram-positive bacteria present) may also be included in the defined microbial profiles for each dermatologic and/or respiratory disease state. Detection of one or more gram-positive bacteria (or, obtaining a ratio or percentage of one or more of these gram-positive bacteria) in the oral microbiome of a cat may enable the systems and methods to indicate or diagnosis the cat as suffering from a dermatologic or respiratory disease.

The same process (comparison to the healthy cohort, PLR transformations, z-test, etc.) was performed for the asthma cohort. FIG. 6 illustrates a distribution of the average log ratio difference scores between pairwise microbial interactions associated with healthy cohorts and the asthmatic cohort. FIG. 7 illustrates sensitivity and specificity of the feline respiratory health test based on a 2-component Gaussian mixture model.

It is important to note that the use of the word ‘predictive’ is not meant to be interpreted as ‘causative’, it simply reflects the fact that a microbe has a significantly different compositional abundance in a particular dermatologic or respiratory condition compared to control. This could either mean that the microbe has an active role in the disease's pathology or that the changes of its compositional abundance are a byproduct of pathology. In either scenario, presence of the microbe in a specific abundance relative to other microbes directly correlates with a dermatologic or respiratory disease state.

The algorithms and disclosed methods of identifying predictive microbes may be continually evolving. A set or grouping of identified predictive microbes may slightly change and evolve as the populations of the cohorts (healthy cats and cats suffering from dermatologic conditions) change and evolve. As more information becomes available regarding microbes and their presence or contribution to a dermatologic or respiratory disease state, the set of identified predictive microbes will change and evolve. The new set of identified predictive microbes may not be 100% different from the initial set, rather a variance of approximately 25% to 85% may be expected. For example, the new set of identified predictive microbes may be 30, 35, 40, 45, 50, 55, 60, 65, 70, 75, or 80% different from the initial set of identified predictive microbes, or a variance defined by any two of the foregoing values. As more cats are added to the cohorts, the set of identified predictive microbes will change and evolve.

### Sequencing and Extraction Protocols

At least one oral swab of a cat may be taken to provide a sample for testing. The oral swabs may target the gum lines of the animal (top and bottom) and/or target the entire mouth of the animal. Microbial DNA may be extracted from the oral swab samples in order to identify which microbial species, and in what relative abundance, are present in the cat's oral microbiome.

Metagenomic DNA may be extracted from the oral samples via heat treatment for approximately one hour on a shaker, with or without bead-beating or the addition of detergents and protein degradation reagents such as proteinase K. In some embodiments, the oral samples are heat treated at approximately 45° C. to 75° C., such as 50° C., 55° C., 60° C., 65° C., 70° C. or within a range defined by any two of the foregoing values.

The gold standard for the comprehensive study of the microbiome is shotgun metagenomic sequencing, which allows capturing complete or near-complete genomes of organisms across all domains of life, not just bacteria and archaea. The gold standard for metagenomic DNA extraction includes a process called bead-beating. It is recommended for complete microbial cell lysis when studying the abundance and composition of the microbiome. The process helps break apart thicker cell walls, such as those of gram-positive bacteria. It is achieved by rapidly agitating samples with grinding media (balls or beads) in a bead beater.

In some exemplary embodiments, the disclosed systems and methods do not use bead-beating for metagenomic DNA extraction and purposefully abandon such a process. The reason for this is that bead-beating can also introduce significant DNA degradation that interferes with downstream sample processing and can therefore lower the quality of the generated metagenomic sequencing library. Since the disclosed systems and methods, according to one embodiment, do not use bead-beating, it is likely that the oral microbiome data in the resulting analyses suffers from under-representation of gram-positive bacteria. Nonetheless, it enables the recognition of disease-characteristic patterns.

After heat treatment of the oral sample, metagenomic DNA may be extracted by SPRI magnetic beads-based DNA extraction (MCLAB, MBC-200) using 80% ethanol for purification. The DNA may be quantified using a GloMax Plate Reader (Promega). Following metagenomic DNA extraction and quantification, the oral samples may be prepared for NGS using the LOTUS DNA library prep kit (IDT), the Next Ultra II FS DNA library prep kit (NEB), or another ligation or tagmentation based DNA library prep kit, following the manufacturer's instructions. The oral samples may be dual-barcoded with iTRU indices. The prepared sequencing libraries may be quantified using a GloMax Plate Reader (Promega) and equal-mass pooled into 96-sample pools. The pools may then be visualized (to assess fragment size distribution) and quantified using a 2100 Bioanalyzer instrument (Agilent). Following standard QC steps, the 96-sample pools may be loaded onto an Illumina HiSeq X or NovaSeq 6000 Next Generation Sequencing machine.

The raw sequencing data may be demultiplexed and trimmed to remove low-quality data using, for example, the program Trimmomatic 0.32. The data may then be mapped to the latest version of, for example, the feline genome Felis_catus_9.0. For every oral sample, there may be approximately 5-7% sequencing reads that do not map to the feline genome. The unmapped reads may be classified using the KRAKEN2 metagenomic sequence classifier (or a suitable alternative) to identify the microbial organisms present in each sample. Bracken, a statistical method for calculating species abundance in DNA sequencing data from a metagenomic sample, may be used on the sequenced data in conjunction with the KRAKEN2 analysis. Bracken may output species level read counts. Based on the outcome of the KRAKEN2 metagenomic sequence classifier and the Bracken calculations, an oral microbial profile for the cat may be generated. The oral microbial profile generated may include data regarding the identity of the microbial species present as well as their relative abundance. The oral microbial profile generated may also include data regarding the relative abundance (increased or decreased) of the microbial species present, as well as a percentage of gram-positive bacteria present.

A confidence score of approximately 0.1 (e.g., 0.08 to 0.15) may be used as a cutoff (or threshold value) for the KRAKEN2 classification algorithm. All samples with fewer than 10,000 classified microbial reads or more than 500,000 classified microbial reads may be filtered out. The reads for microbial species with a non-zero mean of fewer than 10 reads may also be filtered out.

### Methods of Indication and Comparison

Indication of whether a cat is suffering from a dermatologic or respiratory disease relies on a comparison of the cat's current oral microbiome state to the oral microbiomes of cats reported by their pet owners to have been diagnosed by a veterinarian with asthma, atopic dermatitis, food allergic dermatitis, flea allergic dermatitis, or environmental allergic dermatitis. The comparison is based on the compositional abundance of microbes determined by the analysis to be predictive of each of the dermatologic or respiratory conditions.

Computational analysis of the compositional abundance of different microbes present in the oral microbiome involves comparison of the oral sample against a database of samples from cats known to suffer from different dermatologic or respiratory conditions, as well as cats who do not suffer from any known dermatologic or respiratory conditions. In other words, the computational analysis compares the oral microbiome identified/obtained from the oral swab sample to the defined microbial profiles contained in the reference database (discussed more fully above).

In some embodiments, a method for indicating dermatologic or respiratory disease in cats includes receiving an oral swab sample taken from a cat; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample and in what proportions (i.e., abundance), wherein identifying the specific one or more microbes and their abundances results in generation of an oral microbial profile for the cat; and comparing the oral microbial profile for the cat against a database of defined microbial profiles, wherein the database identifies correlations between (i) profiles that include one or more microbes, and (ii) corresponding dermatologic or respiratory diseases.

Based on a result of comparing the oral microbial profile for the cat against the database of defined microbial profiles, the method may further include generating a risk score indicating a likelihood that the cat has a specific dermatologic or respiratory disease. The risk score may be correlated to a stage or severity of the disease state.

In some embodiments, a method for indicating dermatologic or respiratory disease in cats includes receiving an oral swab sample taken from a cat; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample, wherein identifying the specific one or more microbes and their abundance results in generation of an oral microbial profile for the cat.

The method may further include comparing the oral microbial profile for the cat against a database of defined microbial profiles, wherein the database identifies correlations between (i) profiles that include one or more microbes, and (ii) corresponding dermatologic or respiratory diseases; based on a result of comparing the oral microbial profile against the database of defined microbial profiles, generating a risk score indicating a likelihood that the cat has a specific dermatologic or respiratory disease; and in response to generating the risk score and identifying the specific dermatologic or respiratory disease, administering a therapeutic treatment designed to treat the specific dermatologic or respiratory disease. In some embodiments, the therapeutic treatment may be an at-home protocol.

In some embodiments, the therapeutic treatment may include administering a therapeutic compound, such as a compound designed to inhibit or encourage growth of a specific one or more microbial species present in the oral microbiome of the cat. In some embodiments, the therapeutic compound includes a pre-biotic, a post-biotic, a pro-biotic, a medicament or a combination thereof. In some embodiments, the therapeutic compound may include an antibiotic, a corticosteroid, a bronchodilator, or a combination thereof. In some embodiments, the therapeutic treatment includes a topical treatment or bath to help alleviate itching and promote healing of skin.

In some embodiments, the therapeutic treatment or at-home care protocol can alter the composition of the oral microbiome of the cat directly or as a byproduct of the treatment of a specific dermatologic or respiratory disease. In some embodiments, altering the composition of the cat's oral microbiome treats and/or addresses the specific dermatologic or respiratory disease. In some embodiments, the therapeutic treatment repairs the cat's oral microbiome. In some embodiments, repairing the cat's oral microbiome brings the cat's oral microbiome more in line with the oral microbiome (or defined oral microbial profile) of a healthy cat-both in terms of the specific one or more microbial species present and their relative abundance. In some embodiments, the therapeutic treatment protocol is designed to maintain the composition of the oral microbiome of the cat. In some embodiments, the therapeutic treatment protocol is designed to stimulate a metabolic output of the cat's oral microbiome. Stimulating a metabolic output of the cat's oral microbiome may include using known enzymatic pathway analysis tools to provide an additional dimension to the existing microbial composition data to further characterize disease signatures and improve predictive disease models.

### Examples

To start building computational dermatologic and respiratory disease classification algorithms, Pairwise Log-Ratio (PLR) transformation was performed on the Bracken output species level read counts. Next, the significant PLR comparisons (a threshold p-value <0.01) were identified between the control and a condition by performing a z-test. The transformed data may be stored in the reference database. The healthy cohort was compared to the AD, FAD, FLAD and EAD cohorts. The healthy cohort was also compared to the asthma cohort. (See FIGS. 6-7).

The frequency of each microbial species in all significant PLRs was assessed. Only microbial species where 50% or more of their maximum possible comparisons with other species were significant were kept. This measure was used as a proxy for the importance of different microbial species in the four dermatologic and one respiratory condition of interest. These microbial species are “predictive microbial species” for each dermatologic or respiratory condition.

In order to identify population-wide microbial compositional abundance patterns characteristic of asthma, atopic dermatitis, food allergic dermatitis, flea allergic dermatitis and environmental allergic dermatitis, for each of the conditions, each sample was scored by comparing the predictive pairwise log-ratios (pPLRs) of the sample to the mean pPLRs of controls, taking into account the direction and magnitude of the difference. FIGS. 2A-2D illustrate a distribution of the average log ratio difference scores between pairwise microbial interactions associated with atopic dermatitis and healthy controls, food allergic dermatitis and healthy controls, flea allergic dermatitis and healthy controls, and environmental allergic dermatitis and healthy controls. FIG. 6 illustrates a distribution of the average log ratio difference scores between pairwise microbial interactions associated with asthma and healthy cohorts.

Next, we fitted five (5) Gaussian mixture models (one for each dermatologic and respiratory condition) with two (2) components each—healthy cohort and dermatologic or respiratory condition-onto the distribution of the average log ratio difference score between pairwise microbial interactions. This modeling approach generates a 0 to 1 score for each sample, which represents the probability that the sample belongs to the control cohort or to the respective dermatologic condition cohort. FIGS. 3A-3D plot the probability that samples belonging to four of the dermatologic disease cohorts and the control samples would be classified as belonging to their respective cohorts based on each sample's compositional abundance of predictive microbes. A bimodal probability distribution consistent with sample identity was observed between dermatologic condition and control in all cases. In all four instances, there was a minority of disease samples forming a small peak closer to 0 and a small set of control samples forming a slight peak closer to 1. FIG. 7 plots the probability that samples belong to the respiratory disease cohort and the control samples would be classified as belonging to their respective cohorts based on each sample's compositional abundance of predictive microbes.

This suggests that it is possible that a small proportion of cats in the dermatologic or respiratory disease cohorts might actually be healthy or in remission (due to old, wrong or incomplete health information provided by the pet owner), while some cats in the control cohorts could be suffering from a dermatologic or respiratory condition that has not yet been diagnosed or noticed. The sensitivity (ability to detect cats known to suffer from a dermatologic or respiratory condition) and specificity (ability to detect cats in the control cohort as not suffering from a dermatologic or respiratory condition) of the risk classification method for each dermatologic and respiratory condition was tested (see FIGS. 3A-3D and 7). The method's sensitivity is highest for flea allergic dermatitis and lowest for environmental allergic dermatitis, while the specificity is highest for asthma and atopic dermatitis, and lowest for food allergic dermatitis.

Even though a sizable domestic cat cohort (n=4,162) was used to develop the reference database, the health history data for these cats was provided by the pet owner. Despite the fact that pet owners were asked if their cats had been diagnosed by a veterinarian with asthma, atopic dermatitis, food allergic dermatitis, environmental allergic dermatitis, or flea allergic dermatitis, some of the diagnostic precision would have, undoubtedly, suffered, having been relayed by the pet owner. To alleviate this problem and limit instances where a cat reported by their pet owner to be healthy (i.e., not suffering from any known systemic or dermatologic or respiratory conditions) had actually started developing a yet undiagnosed disease, an age limit was set to the control healthy cohort of 1-3 years. This limit was set due to the well-established connection between age and dermatologic, respiratory and systemic disease. Cats below one year of age were intentionally excluded from this group with the purpose of avoiding any potential kitten-specific oral microbiome bias. This segmentation approach comes with the caveat that the healthy control cohort could potentially be biased towards the oral microbiomes of younger cats and not be fully representative of older cats with no dermatologic, respiratory, or systemic diseases.

Though an age limit was set to the control healthy cohort, some embodiments of the present disclosure do not set an age limit. In some embodiments, age of the cat is included as a factor in identifying the cat's risk for having or developing a dermatologic or respiratory disease condition. In some embodiments, age may impact the grouping of the cohorts, with older cats being in a separate cohort from younger cats, even for the same dermatologic or respiratory condition. In some embodiments, age is a factor applied to a cat's risk assessment after comparison of the cat's oral microbial profile to the cohorts (healthy and pathological). In some embodiments, age is incorporated into the oral microbial profile obtained and generated for the cat.

In addition to the foregoing, microbes identified as associated with or predictive for a dermatologic condition may be further predictive for stages or grades of the dermatologic or respiratory condition.

The risk score generation methodology disclosed herein is based on oral microbiome compositional analysis. Other embodiments of the disclosed methods may also include incorporating predictions of the metabolic output of the oral microbiome (generated by enzymatic pathway analysis tools or metabolomics), alongside the oral microbiome compositional abundance analysis for the purpose of predictive risk of dermatologic or respiratory conditions.

### Additional Terms and Definitions

Unless defined otherwise, all technical and scientific terms used herein have the same meaning as commonly understood by one of ordinary skill in the art to which the present disclosure pertains.

Various “aspects” of the present disclosure, including systems, methods, and/or products may be illustrated with reference to one or more “embodiments,” which are exemplary in nature. As used herein, the terms “aspect” and “embodiment” may be used interchangeably. The term “embodiment” can also mean “serving as an example, instance, or illustration,” and should not necessarily be construed as preferred or advantageous over other aspects disclosed herein. In addition, reference to an “embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

“Treating” or “treatment” as used herein covers the treatment of the disease or condition of interest in a cat, having the disease or condition of interest, and includes: (i) preventing the disease or condition from occurring in a cat, in particular, when such cat is actually starting to develop the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Tables

TABLE 1
Predictive microbes for atopic dermatitis (AD), food allergic dermatitis (FAD), flea allergic
dermatitis (FLAD) and environmental allergic dermatitis (EAD).

| AD | FAD | FLAD | EAD |
|---|---|---|---|
| Frederiksenia | Frederiksenia | Frederiksenia | Frederiksenia |
| canicola: 123824 | canicola: 123824 | canicola: 123824 | canicola: 123824 |
| Avibacterium | Avibacterium | Streptobacillus | Streptobacillus |
| paragallinarum: 728 | paragallinarum: 728 | moniliformis DSM | moniliformis DSM |
| | | 12112: 34105 | 12112: 34105 |
| Pasteurella | Pasteurella | Avibacterium | Pasteurella |
| dagmatis: 754 | dagmatis: 754 | paragallinarum: 728 | dagmatis: 754 |
| Streptobacillus | Streptobacillus | Pasteurella | Avibacterium |
| moniliformis DSM | moniliformis DSM | dagmatis: 754 | paragallinarum: 728 |
| 12112: 34105 | 12112: 34105 | | |
| Glaesserella sp. 15- | Haemophilus | Haemophilus | Glaesserella sp. 15- |
| 184: 2030797 | haemolyticus: 726 | haemolyticus: 726 | 184: 2030797 |
| Haemophilus | Glaesserella sp. 15- | Glaesserella sp. 15- | Haemophilus |
| haemolyticus: 726 | 184: 2030797 | 184: 2030797 | haemolyticus: 726 |
| Conchiformibius | Conchiformibius | Moraxella catarrhalis | Conchiformibius |
| steedae: 153493 | steedae: 153493 | BBH18: 480 | steedae: 153493 |
| Moraxella catarrhalis | Moraxella catarrhalis | Neisseria weaveri: 28091 | Moraxella catarrhalis |
| BBH18: 480 | BBH18: 480 | | BBH18: 480 |
| Neisseria | Moraxella | Conchiformibius | Moraxella |
| zoodegmatis: 326523 | cuniculi: 34061 | steedae: 153493 | bovoculi: 386891 |
| Moraxella | Neisseria | Neisseria | Moraxella |
| cuniculi: 34061 | zoodegmatis: 326523 | zoodegmatis: 326523 | cuniculi: 34061 |
| Neisseria | Moraxella | Fusobacterium | Neisseria |
| animaloris: 326522 | bovoculi: 386891 | pseudoperiodonticum: | zoodegmatis: 326523 |
| | | 2663009 | |
| Moraxella | Neisseria | Moraxella | Histophilus somni |
| bovoculi: 386891 | animaloris: 326522 | cuniculi: 34061 | 2336: 731 |
| Neisseria weaveri: 28091 | Neisseria weaveri: 28091 | Capnocytophaga | Neisseria weaveri: 28091 |
| | | canimorsus Cc5: 28188 | |
| Neisseria | Moraxella ovis: 29433 | Fusobacterium | Moraxella ovis: 29433 |
| musculi: 1815583 | | hwasookii ChDC | |
| | | F300: 1583098 | |
| Neisseria | Moraxella | Fusobacterium sp. oral | Fusobacterium sp. oral |
| wadsworthii: 607711 | osloensis: 34062 | taxon 203: 671211 | taxon 203: 671211 |
| Moraxella ovis: 29433 | Saccharomyces | Neisseria | Neisseria |
| | cerevisiae S288C: 4932 | animaloris: 326522 | animaloris: 326522 |
| Moraxella | Neisseria | Moraxella | Moraxella |
| osloensis: 34062 | wadsworthii: 607711 | bovoculi: 386891 | osloensis: 34062 |
| Histophilus somni | Histophilus somni | Histophilus somni | Capnocytophaga |
| 2336: 731 | 2336: 731 | 2336: 731 | canimorsus Cc5: 28188 |
| Neisseria canis: 493 | Neisseria | Neisseria | Fusobacterium |
| | musculi: 1815583 | wadsworthii: 607711 | pseudoperiodonticum: |
| | | | 2663009 |
| Cutibacterium acnes | Fusobacterium sp. oral | Moraxella ovis: 29433 | Saccharomyces |
| subsp. defendens ATCC | taxon 203: 671211 | | cerevisiae S288C: 4932 |
| 11828: 1747 | | | |
| Pseudomonas sp. | Neisseria canis: 493 | Capnocytophaga sp. | Parvimonas |
| TKP: 1415630 | | H4358: 1945658 | micra: 33033 |
| Saccharomyces | Saccharomyces | Capnocytophaga sp. | Fusobacterium |
| cerevisiae S288C: 4932 | eubayanus: 1080349 | H2931: 1945657 | hwasookii ChDC |
| | | | F300: 1583098 |
| Fusobacterium | Capnocytophaga | Neisseria canis: 493 | Capnocytophaga sp. |
| pseudoperiodonticum: | canimorsus Cc5: 28188 | | H4358: 1945658 |
| 2663009 | | | |
| Neisseria | Salmonella enterica | Fusobacterium | Capnocytophaga sp. |
| dentiae: 194197 | subsp. salamae serovar | periodonticum: 860 | H2931: 1945657 |
| | 57: z29: z42: 28901 | | |
| Capnocytophaga sp. | Fusobacterium | Neisseria | Streptococcus |
| H4358: 1945658 | pseudoperiodonticum: | musculi: 1815583 | dysgalactiae subsp. |
| | 2663009 | | equisimilis RE378: 1334 |
| Capnocytophaga sp. | Alloprevotella sp. | Pasteurella multocida | Neisseria |
| H2931: 1945657 | E39: 2133944 | subsp. septica: 747 | wadsworthii: 607711 |
| Capnocytophaga | Fusobacterium | Moraxella | Neisseria |
| canimorsus Cc5: 28188 | hwasookii ChDC | osloensis: 34062 | musculi: 1815583 |
| | F300: 1583098 | | |
| Neisseria | Capnocytophaga sp. | Acinetobacter johnsonii | Neisseria canis: 493 |
| shayeganii: 607712 | H4358: 1945658 | XBB1: 40214 | |
| Pasteurella multocida | Pasteurella multocida | Fusobacterium | Leptotrichia sp. oral |
| subsp. septica: 747 | subsp. septica: 747 | nucleatum subsp. | taxon 212: 712357 |
| | | vincentii ChDC F8: 851 | |
| Actinomyces | Cutibacterium acnes | Capnocytophaga | Streptococcus oralis |
| oris: 544580 | subsp. defendens ATCC | cynodegmi: 28189 | subsp. tigurinus: 1303 |
| | 11828: 1747 | | |
| Parvimonas | Capnocytophaga sp. | Neisseria | Pasteurella multocida |
| micra: 33033 | H2931: 1945657 | shayeganii: 607712 | subsp. septica: 747 |
| Psychrobacter sp. | Lautropia | Parvimonas | Lachnoanaerobaculum |
| PRwf-1: 349106 | mirabilis: 47671 | micra: 33033 | umeaense: 617123 |
| Streptomyces sp. GBA | Streptococcus | Streptococcus | Porphyromonas |
| 94-10: 1218177 | dysgalactiae subsp. | dysgalactiae subsp. | asaccharolytica DSM |
| | equisimilis RE378: 1334 | equisimilis RE378: 1334 | 20707: 28123 |
| [Brevibacterium] flavum | Parvimonas | Leptotrichia sp. oral | Fungi: Basidiomycota: |
| ZL-1: 92706 | micra: 33033 | taxon 212: 712357 | Malassezia restricta: 76775 |
| Pseudomonas sp. EGD- | Neisseria | Burkholderia mallei | Fusobacterium |
| AKN5: 1524461 | shayeganii: 607712 | SAVP1: 13373 | nucleatum subsp. |
| | | | vincentii ChDC F8: 851 |
| Tessaracoccus | Acinetobacter johnsonii | Campylobacter sp. | Dichelobacter nodosus |
| lapidicaptus: 1427523 | XBB1: 40214 | CFSAN093260: 2572085 | VCS1703A: 870 |
| Xanthomonas perforans | Porphyromonas | Campylobacter sp. | Streptococcus equi |
| 91-118: 442694 | asaccharolytica DSM | CFSAN093256: 2572082 | subsp. zooepidemicus |
| | 20707: 28123 | | MGCS10565: 1336 |
| Arcobacter thereius | Dichelobacter nodosus | Klebsiella sp. WP4- | Porphyromonas |
| LMG 24486: 544718 | VCS1703A: 870 | W18-ESBL-05: 2675713 | cangingivalis: 36874 |
| Capnocytophaga | Porphyromonas | Dietzia sp. DQ12-45- | Saccharomyces |
| stomatis: 1848904 | cangingivalis: 36874 | 1b: 912801 | eubayanus: 1080349 |
| Streptococcus | Neisseria | Brucella abortus bv. 9 | Alloprevotella sp. |
| intermedius JTH08: 1338 | dentiae: 194197 | str. C68: 235 | E39: 2133944 |
| Porphyromonas | Streptococcus equi | Staphylococcus | Acinetobacter johnsonii |
| crevioricanis: 393921 | subsp. zooepidemicus | piscifermentans: 70258 | XBB1: 40214 |
| | MGCS10565: 1336 | | |
| Chryseobacterium | Prevotella fusca JCM | Brevibacterium sp. | Streptococcus |
| gallinarum: 1324352 | 17724: 589436 | PAMC23299: 2762330 | canis: 1329 |
| Xanthomonas | Psychrobacter sp. | Tessaracoccus | Capnocytophaga |
| translucens pv. | PRwf-1: 349106 | lapidicaptus: 1427523 | cynodegmi: 28189 |
| undulosa: 343 | | | |
| Prevotella oris: 28135 | Fusobacterium | Pseudomonas sp. | Campylobacter sp. |
| | nucleatum subsp. | FDAARGOS_761: | CCUG 57310: 2517362 |
| | vincentii ChDC F8: 851 | 2545800 | |
| Bergeyella | Leptotrichia sp. oral | Arcobacter thereius | Fusobacterium |
| cardium: 1585976 | taxon 212: 712357 | LMG 24486: 544718 | necrophorum subsp. |
| | | | necrophorum: 859 |
| Aeromonas salmonicida | Psychrobacter sp. | Corynebacterium | Neisseria |
| subsp. smithia: 645 | P2G3: 1699622 | sanguinis: 2594913 | shayeganii: 607712 |
| Candidatus | Lachnoanaerobaculum | Chryseobacterium | Acinetobacter lwoffii |
| Nanosynbacter | umeaense: 617123 | gallinarum: 1324352 | WJ10621: 28090 |
| lyticus: 2093824 | | | |
| Clostridioides difficile | Psychrobacter sp. | Cardiobacterium | Streptococcus |
| R20291: 1496 | P11G5: 1699624 | hominis: 2718 | pseudoporcinus: 361101 |
| Tannerella forsythia | Streptococcus | Clostridioides difficile | Klebsiella sp. |
| KS16: 28112 | canis: 1329 | R20291: 1496 | MPUS7: 2697371 |
| Stenotrophomonas | Shigella sonnei: 624 | Bacteroides sp. | Enterobacter sp. |
| acidaminiphila: 128780 | | A1C1: 2528203 | CRENT-193: 2051905 |
| Dermabacter | Capnocytophaga | Stenotrophomonas | Streptomyces sp. S1D4- |
| jinjuensis: 1667168 | cynodegmi: 28189 | nitritireducens: 83617 | 14: 2594461 |
| Treponema sp. OMZ | Yersinia pestis biovar | Tannerella forsythia | Klebsiella sp. WP3- |
| 804: 120683 | Medievalis str. Harbin | KS16: 28112 | W18-ESBL-02: 2675710 |
| | 35: 632 | | |
| Stenotrophomonas | Klebsiella sp. | Treponema pedis str. | Citrobacter freundii |
| nitritireducens: 83617 | MPUS7: 2697371 | TA4: 409322 | complex sp. |
| | | | CFNIH3: 2077147 |
| Campylobacter | Streptomyces sp. | Flavonifractor | Pseudomonas sp. EGD- |
| rectus: 203 | ICC4: 2099584 | plautii: 292800 | AKN5: 1524461 |
| Porphyromonas | Streptomyces sp. S1D4- | Bergeyella | Salmonella sp. |
| gingivalis W83: 837 | 14: 2594461 | cardium: 1585976 | S13: 2686305 |
| Comamonas | Citrobacter sp. | Stenotrophomonas | Shigella boydii |
| aquatica: 225991 | RHBSTW- | acidaminiphila: 128780 | Sb227: 621 |
| | 00229: 2742641 | | |
| Delftia | Citrobacter sp. | Pseudopropionibacterium | Aeromonas sp. |
| tsuruhatensis: 180282 | RHBSTW- | propionicum | ASNIH7: 1920107 |
| | 00570: 2742655 | F0230a: 1750 | |
| Treponema sp. OMZ | Pseudomonas sp. EGD- | Ottowia sp. oral taxon | Pseudomonas sp. |
| 838: 1539298 | AKN5: 1524461 | 894: 1658672 | FDAARGOS_761: |
| | | | 2545800 |
| Pseudomonas | Citrobacter sp. | Candidatus | Xanthomonas perforans |
| denitrificans | RHBSTW- | Nanosynbacter | 91-118: 442694 |
| (nom. rej.): 43306 | 01044: 2742678 | lyticus: 2093824 | |
| Ottowia sp. oral taxon | Klebsiella sp. WP3- | Bacteroides | Actinomyces sp. oral |
| 894: 1658672 | W18-ESBL-02: 2675710 | cellulosilyticus: 246787 | taxon 171 str. |
| | | | F0337: 706438 |
| Prevotella denticola | Serratia sp. LS- | Xanthomonas | Flavonifractor |
| F0289: 28129 | 1: 2485839 | translucens pv. | plautii: 292800 |
| | | undulosa: 343 | |
| Comamonas sp. NLF-7- | Bacteroides sp. HF- | Campylobacter sp. | Xanthomonas |
| 7: 2597701 | 162: 2785531 | RM16192: 1660080 | euroxanthea: 2259622 |
| Desulfovibrio sp. | Pseudomonas sp. | Prevotella denticola | Corynebacterium |
| G11: 631220 | FDAARGOS_761: 2545800 | F0289: 28129 | mycetoides: 38302 |
| Bacteroides sp. | Burkholderia sp. | Bacteroides | Arcobacter thereius |
| A1C1: 2528203 | 2002721687: 1468409 | uniformis: 820 | LMG 24486: 544718 |
| Bacteroides | Xanthomonas perforans | Comamonas | Actinomyces sp. oral |
| caccae: 47678 | 91-118: 442694 | aquatica: 225991 | taxon 169: 712116 |
| Melaminivora sp. SC2- | Actinomyces sp. oral | Desulfovibrio sp. | Tannerella forsythia |
| 9: 2109913 | taxon 169: 712116 | G11: 631220 | KS16: 28112 |
| Diaphorobacter | Corynebacterium | Prevotella oris: 28135 | Pseudomonas sp. |
| polyhydroxybutyrativorans: | mycetoides: 38302 | | TKP: 1415630 |
| 1546149 | | | |
| Bacteroides | Actinomyces sp. oral | Melaminivora sp. SC2- | Prevotella oris: 28135 |
| intestinalis: 329854 | taxon 171 str. | 9: 2109913 | |
| | F0337: 706438 | | |
| Bacteroides fragilis | Xanthomonas | Bacteroides | Bacteroides sp. |
| YCH46: 817 | euroxanthea: 2259622 | heparinolyticus: 28113 | A1C1: 2528203 |
| Bacteroides | Arcobacter thereius | Ottowia oryzae: 2109914 | Aeromonas salmonicida |
| uniformis: 820 | LMG 24486: 544718 | | subsp. smithia: 645 |
| [Arcobacter] | Bacteroides sp. | [Arcobacter] | [Arcobacter] |
| porcinus: 1935204 | A1C1: 2528203 | porcinus: 1935204 | porcinus: 1935204 |
| Bacteroides | Treponema pedis str. | Aeromonas sp. | Bacteroides |
| cellulosilyticus: 246787 | TA4: 409322 | ASNIH3: 1636608 | cellulosilyticus: 246787 |
| Acidovorax | Streptococcus | Delftia | Prevotella denticola |
| carolinensis: 553814 | intermedius JTH08: 1338 | tsuruhatensis: 180282 | F0289: 28129 |
| Acidovorax sp. | Flavonifractor | Bacteroides | Treponema sp. OMZ |
| T1: 1858609 | plautii: 292800 | intestinalis: 329854 | 838: 1539298 |
| Ralstonia | Campylobacter sp. | Comamonas sp. NLF-7- | Treponema sp. OMZ |
| mannitolilytica: 105219 | RM16192: 1660080 | 7: 2597701 | 804: 120683 |
| Pseudopropionibacterium | Bergeyella | Ralstonia | Bacteroides |
| propionicum | cardium: 1585976 | mannitolilytica: 105219 | heparinolyticus: 28113 |
| F0230a: 1750 | | | |
| Ottowia oryzae: 2109914 | Prevotella oris: 28135 | Bacteroides | Cardiobacterium |
| | | caccae: 47678 | hominis: 2718 |
| Bacteroides | Tannerella forsythia | Bacteroides fragilis | Desulfovibrio sp. |
| heparinolyticus: 28113 | KS16: 28112 | YCH46: 817 | G11: 631220 |
| Alicycliphilus | Stenotrophomonas | Diaphorobacter | Bacteroides fragilis |
| denitrificans | acidaminiphila: 128780 | polyhydroxybutyrativorans: | YCH46: 817 |
| K601: 179636 | | 1546149 | |
| Bacteroides | Aeromonas salmonicida | Pseudomonas | Lysobacter |
| caecimuris: 1796613 | subsp. smithia: 645 | denitrificans | oculi: 2698682 |
| | | (nom. rej.): 43306 | |
| Acidovorax ebreus | Treponema | Dermabacter | Bacteroides |
| TPSY: 721785 | putidum: 221027 | jinjuensis: 1667168 | uniformis: 820 |
| Diaphorobacter sp. | Treponema denticola | Treponema denticola | Comamonas sp. NLF-7- |
| JS3050: 2735554 | OTK: 158 | OTK: 158 | 7: 2597701 |
| Desulfomicrobium orale | Prevotella denticola | Acidovorax | Dermabacter |
| DSM 12838: 132132 | F0289: 28129 | carolinensis: 553814 | jinjuensis: 1667168 |
| Bacteroides | Candidatus | Bacteroides | Ottowia sp. oral taxon |
| zoogleoformans: 28119 | Nanosynbacter | xylanisolvens: 371601 | 894: 1658672 |
| | lyticus: 2093824 | | |
| Bacteroides | [Arcobacter] | Alicycliphilus | Bacteroides |
| xylanisolvens: 371601 | porcinus: 1935204 | denitrificans | intestinalis: 329854 |
| | | K601: 179636 | |
| Desulfobulbus | Treponema sp. OMZ | Treponema | Bacteroides |
| oralis: 1986146 | 804: 120683 | phagedenis: 162 | caccae: 47678 |
| | Lysobacter | Treponema | Comamonas |
| | oculi: 2698682 | putidum: 221027 | aquatica: 225991 |
| | Treponema sp. OMZ | Treponema sp. OMZ | Xanthomonas |
| | 838: 1539298 | 804: 120683 | translucens pv. |
| | | | undulosa: 343 |
| | Bacteroides | Aeromonas salmonicida | Porphyromonas |
| | uniformis: 820 | subsp. smithia: 645 | gingivalis W83: 837 |
| | Aeromonas sp. | Acidovorax ebreus | Melaminivora sp. SC2- |
| | ASNIH3: 1636608 | TPSY: 721785 | 9: 2109913 |
| | Bacteroides | Acidovorax sp. | Pseudopropionibacterium |
| | heparinolyticus: 28113 | JS42: 232721 | propionicum |
| | | | F0230a: 1750 |
| | Bacteroides | Porphyromonas | Stenotrophomonas |
| | cellulosilyticus: 246787 | gingivalis W83: 837 | acidaminiphila: 128780 |
| | Desulfovibrio sp. | Treponema sp. OMZ | Ottowia oryzae: 2109914 |
| | G11: 631220 | 838: 1539298 | |
| | Delftia | Bacteroides | Aeromonas sp. |
| | tsuruhatensis: 180282 | caecimuris: 1796613 | ASNIH3: 1636608 |
| | Acidovorax | Desulfobulbus | Delftia |
| | carolinensis: 553814 | oralis: 1986146 | tsuruhatensis: 180282 |
| | Cardiobacterium | Acidovorax sp. | Diaphorobacter |
| | hominis: 2718 | T1: 1858609 | polyhydroxybutyrativorans: |
| | | | 1546149 |
| | Comamonas sp. NLF-7- | Bacteroides | Stenotrophomonas |
| | 7: 2597701 | zoogleoformans: 28119 | nitritireducens: 83617 |
| | Stenotrophomonas | Desulfomicrobium orale | Acidovorax |
| | nitritireducens: 83617 | DSM 12838: 132132 | carolinensis: 553814 |
| | Ottowia sp. oral taxon | Diaphorobacter sp. | Desulfomicrobium orale |
| | 894: 1658672 | JS3050: 2735554 | DSM 12838: 132132 |
| | Bacteroides | | Ralstonia |
| | intestinalis: 329854 | | mannitolilytica: 105219 |
| | Porphyromonas | | Alicycliphilus |
| | gingivalis W83: 837 | | denitrificans |
| | | | K601: 179636 |
| | Bacteroides | | Bacteroides |
| | caccae: 47678 | | caecimuris: 1796613 |
| | Comamonas | | Acidovorax sp. |
| | aquatica: 225991 | | T1: 1858609 |
| | Bacteroides fragilis | | Pseudomonas |
| | YCH46: 817 | | denitrificans |
| | | | (nom. rej.): 43306 |
| | Diaphorobacter | | Desulfobulbus |
| | polyhydroxybutyrativorans: | | oralis: 1986146 |
| | 1546149 | | |
| | Dermabacter | | Bacteroides |
| | jinjuensis: 1667168 | | xylanisolvens: 371601 |
| | Melaminivora sp. SC2- | | Acidovorax sp. |
| | 9: 2109913 | | JS42: 232721 |
| | Pseudopropionibacterium | | Acidovorax ebreus |
| | propionicum | | TPSY: 721785 |
| | F0230a: 1750 | | |
| | Ralstonia | | Bacteroides |
| | mannitolilytica: 105219 | | zoogleoformans: 28119 |
| | Ottowia oryzae: 2109914 | | Diaphorobacter sp. |
| | | | JS3050: 2735554 |
| | Xanthomonas | | |
| | translucens pv. | | |
| | undulosa: 343 | | |
| | Pseudomonas | | |
| | denitrificans | | |
| | (nom. rej.): 43306 | | |
| | Acidovorax sp. | | |
| | T1: 1858609 | | |
| | Bacteroides | | |
| | caecimuris: 1796613 | | |
| | Alicycliphilus | | |
| | denitrificans | | |
| | K601: 179636 | | |
| | Acidovorax sp. | | |
| | JS42: 232721 | | |
| | Desulfomicrobium orale | | |
| | DSM 12838: 132132 | | |
| | Desulfobulbus | | |
| | oralis: 1986146 | | |
| | Acidovorax ebreus | | |
| | TPSY: 721785 | | |
| | Bacteroides | | |
| | zoogleoformans: 28119 | | |
| | Bacteroides | | |
| | xylanisolvens: 371601 | | |
| | Diaphorobacter sp. | | |
| | JS3050: 2735554 | | |

TABLE 2
Predictive microbes for asthma.

Frederiksenia canicola: 123824
Avibacterium paragallinarum: 728
Pasteurella dagmatis: 754
Glaesserella sp. 15-184: 2030797
Streptobacillus moniliformis DSM 12112: 34105
Haemophilus haemolyticus: 726
Neisseria zoodegmatis: 326523
Conchiformibius steedae: 153493
Moraxella cuniculi: 34061
Neisseria weaveri: 28091
Neisseria animaloris: 326522
Moraxella bovoculi: 386891
Moraxella catarrhalis BBH18: 480
Moraxella ovis: 29433
Neisseria wadsworthii: 607711
Moraxella osloensis: 34062
Neisseria musculi: 1815583
Saccharomyces cerevisiae S288C: 4932
Histophilus somni 2336: 731
Neisseria canis: 493
Capnocytophaga canimorsus Cc5: 28188
Malassezia restricta: 76775
Capnocytophaga sp. H4358: 1945658
Cutibacterium acnes subsp. defendens ATCC 11828: 1747
Capnocytophaga sp. H2931: 1945657
Pasteurella multocida subsp. septica: 747
Fusobacterium pseudoperiodonticum: 2663009
Fusobacterium sp. oral taxon 203: 671211
Saccharomyces eubayanus: 1080349
Streptococcus dysgalactiae subsp. equisimilis RE378: 1334
Dichelobacter nodosus VCS1703A: 870
Parvimonas micra: 33033
Lautropia mirabilis: 47671
Alloprevotella sp. E39: 2133944
Fusobacterium hwasookii ChDC F300: 1583098
Neisseria dentiae: 194197
Fusobacterium nucleatum subsp. vincentii ChDC F8: 851
Pseudomonas sp. TKP: 1415630
Porphyromonas cangingivalis: 36874
Prevotella fusca JCM 17724: 589436
Leptotrichia sp. oral taxon 212: 712357
Acinetobacter johnsonii XBB1: 40214
Porphyromonas asaccharolytica DSM 20707: 28123
Streptococcus agalactiae NEM316: 1311
Fusobacterium periodonticum: 860
Neisseria shayeganii: 607712
Streptococcus equi subsp. zooepidemicus MGCS10565: 1336
Lachnoanaerobaculum umeaense: 617123
Capnocytophaga cynodegmi: 28189
Viruses: Uroviricota: Serratia phage Moabite: 2587814
Staphylococcus piscifermentans: 70258
Campylobacter sp. CFSAN093260: 2572085
Citrobacter sp. RHBSTW-01044: 2742678
Salmonella sp. SCFS4: 2725417
Citrobacter sp. RHBSTW-00599: 2742657
Citrobacter sp. RHB36-C18: 2742627
Serratia sp. JKS000199: 1938820
Klebsiella sp. WP3-W18-ESBL-02: 2675710
Citrobacter sp. RHBSTW-00229: 2742641
Citrobacter sp. RHBSTW-00570: 2742655
Streptomyces sp. S1D4-14: 2594461
Serratia sp. LS-1: 2485839
Bacteria: Spirochaetes: Treponema pallidum
subsp. pertenue str. SamoaD: 160
Arcobacter thereius LMG 24486: 544718
Xanthomonas perforans 91-118: 442694
Actinomyces sp. oral taxon 169: 712116
Prevotella dentalis DSM 3688: 52227
Flavonifractor plautii: 292800
Corynebacterium mycetoides: 38302
Actinomyces sp. oral taxon 171 str. F0337: 706438
Prevotella oris: 28135
Prevotella denticola F0289: 28129
Bacteroides sp. A1C1: 2528203
Tannerella forsythia KS16: 28112
Aeromonas salmonicida subsp. smithia: 645
Aeromonas sp. ASNIH3: 1636608
[Arcobacter] porcinus: 1935204
Cardiobacterium hominis: 2718
Bacteroides cellulosilyticus: 246787
Desulfovibrio sp. G11: 631220
Stenotrophomonas acidaminiphila: 128780
Delftia tsuruhatensis: 180282
Xanthomonas translucens pv. undulosa: 343
Stenotrophomonas nitritireducens: 83617
Candidatus Nanosynbacter lyticus : 2093824
Comamonas aquatica: 225991
Bacteroides heparinolyticus: 28113
Bacteroides fragilis YCH46: 817
Pseudopropionibacterium propionicum F0230a: 1750
Bacteroides uniformis: 820
Ottowia sp. oral taxon 894: 1658672
Bacteroides intestinalis: 329854
Bacteroides caccae: 47678
Comamonas sp. NLF-7-7: 2597701
Acidovorax carolinensis: 553814
Melaminivora sp. SC2-9: 2109913
Ottowia oryzae: 2109914
Bacteroides caecimuris: 1796613
Dermabacter jinjuensis: 1667168
Porphyromonas gingivalis W83: 837
Diaphorobacter polyhydroxybutyrativorans: 1546149
Acidovorax sp. T1: 1858609
Ralstonia mannitolilytica: 105219
Pseudomonas denitrificans (nom. rej.): 43306
Desulfomicrobium orale DSM 12838: 132132
Desulfobulbus oralis: 1986146
Acidovorax sp. JS42: 232721
Bacteroides xylanisolvens: 371601
Bacteroides zoogleoformans: 28119
Alicycliphilus denitrificans K601: 179636
Diaphorobacter sp. JS3050: 2735554
Acidovorax ebreus TPSY: 721785

TABLE 3
Predictive microbes alongside their taxonomic classification
for asthma. Of the 112 total predictive microbes (see Table 2),
12.5% are gram-positive bacteria.

| | | |
|---|---|---|
| bacteria | proteobacteria | 55.36% |
| bacteria | fusobacteria | 6% |
| bacteria | firmicutes | 6% |
| bacteria | bacteroidetes | 20% |
| bacteria | spirochaetes | 1% |
| bacteria | actinobacteria | 6% |
| bacteria | candidatus | 1% |
| fungi | | 3% |
| viruses | | 1% |

TABLE 4
Predictive microbes alongside their taxonomic classification
for food allergic dermatitis. Of the 122 total predictive microbes
for food allergic dermatitis (see Table 1), approximately 10.66%
are gram-positive bacteria. Note that ‘candidatus’ stands for
well-characterized, but yet uncultured bacteria.

| | | |
|---|---|---|
| bacteria | proteobacteria | 57% |
| bacteria | fusobacteria | 5% |
| bacteria | firmicutes | 6% |
| bacteria | bacteroidetes | 20% |
| bacteria | spirochaetes | 4% |
| bacteria | actinobacteria | 7% |
| bacteria | candidatus | 1% |
| fungi | | 2% |

TABLE 5
Predictive microbes alongside their taxonomic classification
for flea allergic dermatitis. Of the 99 total predictive microbes
for flea allergic dermatitis (see Table 1), approximately 11.11%
are gram-positive bacteria. Note that ‘candidatus’ stands for
well-characterized, but yet uncultured bacteria.

| | | |
|---|---|---|
| bacteria | proteobacteria | 55% |
| bacteria | fusobacteria | 7% |
| bacteria | firmicutes | 5% |
| bacteria | bacteroidetes | 20% |
| bacteria | spirochaetes | 6% |
| bacteria | actinobacteria | 6% |
| bacteria | candidatus | 1% |
| fungi | | 0% |

TABLE 6
Predictive microbes alongside their taxonomic classification
for atopic dermatitis. Of the 86 total predictive microbes
for atopic dermatitis (see Table 1), approximately 11.63%
are gram-positive bacteria. Note that ‘candidatus’ stands
for well-characterized, but yet uncultured bacteria.

| | | |
|---|---|---|
| bacteria | proteobacteria | 57% |
| bacteria | fusobacteria | 2% |
| bacteria | firmicutes | 3% |
| bacteria | bacteroidetes | 24% |
| bacteria | spirochaetes | 2% |
| bacteria | actinobacteria | 8% |
| bacteria | candidatus | 1% |
| fungi | | 1% |

TABLE 7
Predictive microbes alongside their taxonomic classification
for enviornmental allergic dermatitis. Of the 110 total predictive
microbes for enviornmental allergic dermatitis (see Table 1),
approximately 7.27% are gram-positive bacteria. Note that ‘candidatus’
stands for well-characterized, but yet uncultured bacteria.

| | | |
|---|---|---|
| bacteria | proteobacteria | 57% |
| bacteria | fusobacteria | 6% |
| bacteria | firmicutes | 7% |
| bacteria | bacteroidetes | 19% |
| bacteria | spirochaetes | 2% |
| bacteria | actinobacteria | 5% |
| fungi | | 3% |

TABLE 8
The relative increased or decreased abundance for each
predictive microbe for asthma.

| | Increase/ |
|---|---|
| | decreased |
| | relative |
| Microbe | abundance |
| Frederiksenia canicola: 123824 | decreased |
| Avibacterium paragallinarum: 728 | decreased |
| Pasteurella dagmatis: 754 | decreased |
| Glaesserella sp. 15-184: 2030797 | decreased |
| Streptobacillus moniliformis DSM 12112: 34105 | decreased |
| Haemophilus haemolyticus: 726 | decreased |
| Neisseria zoodegmatis: 326523 | decreased |
| Conchiformibius steedae: 153493 | decreased |
| Moraxella cuniculi: 34061 | decreased |
| Neisseria weaveri: 28091 | decreased |
| Neisseria animaloris: 326522 | decreased |
| Moraxella bovoculi: 386891 | decreased |
| Moraxella catarrhalis BBH18: 480 | decreased |
| Moraxella ovis: 29433 | decreased |
| Neisseria wadsworthii: 607711 | decreased |
| Moraxella osloensis: 34062 | decreased |
| Neisseria musculi: 1815583 | decreased |
| Saccharomyces cerevisiae S288C: 4932 | decreased |
| Histophilus somni 2336: 731 | decreased |
| Neisseria canis: 493 | decreased |
| Capnocytophaga canimorsus Cc5: 28188 | decreased |
| Malassezia restricta: 76775 | decreased |
| Capnocytophaga sp. H4358: 1945658 | decreased |
| Cutibacterium acnes subsp. defendens ATCC | decreased |
| 11828: 1747 | |
| Capnocytophaga sp. H2931: 1945657 | decreased |
| Pasteurella multocida subsp. septica: 747 | decreased |
| Fusobacterium pseudoperiodonticum: 2663009 | decreased |
| Fusobacterium sp. oral taxon 203: 671211 | decreased |
| Saccharomyces eubayanus: 1080349 | decreased |
| Streptococcus dysgalactiae subsp. equisimilis | decreased |
| RE378: 1334 | |
| Dichelobacter nodosus VCS1703A: 870 | decreased |
| Parvimonas micra: 33033 | decreased |
| Lautropia mirabilis: 47671 | decreased |
| Alloprevotella sp. E39: 2133944 | decreased |
| Fusobacterium hwasookii ChDC F300: 1583098 | decreased |
| Neisseria dentiae: 194197 | decreased |
| Fusobacterium nucleatum subsp. vincentii ChDC | decreased |
| F8: 851 | |
| Pseudomonas sp. TKP: 1415630 | decreased |
| Porphyromonas cangingivalis: 36874 | decreased |
| Prevotella fusca JCM 17724: 589436 | decreased |
| Leptotrichia sp. oral taxon 212: 712357 | decreased |
| Acinetobacter johnsonii XBB1: 40214 | decreased |
| Porphyromonas asaccharolytica DSM 20707: 28123 | decreased |
| Streptococcus agalactiae NEM316: 1311 | decreased |
| Fusobacterium periodonticum: 860 | decreased |
| Neisseria shayeganii: 607712 | decreased |
| Streptococcus equi subsp. zooepidemicus | decreased |
| MGCS10565: 1336 | |
| Lachnoanaerobaculum umeaense: 617123 | decreased |
| Capnocytophaga cynodegmi: 28189 | decreased |
| Viruses: Uroviricota: Serratia phage Moabite: 2587814 | increased |
| Staphylococcus piscifermentans: 70258 | increased |
| Campylobacter sp. CFSAN093260: 2572085 | increased |
| Citrobacter sp. RHBSTW-01044: 2742678 | increased |
| Salmonella sp. SCFS4: 2725417 | increased |
| Citrobacter sp. RHBSTW-00599: 2742657 | increased |
| Citrobacter sp. RHB36-C18: 2742627 | increased |
| Serratia sp. JKS000199: 1938820 | increased |
| Klebsiella sp. WP3-W18-ESBL-02: 2675710 | increased |
| Citrobacter sp. RHBSTW-00229: 2742641 | increased |
| Citrobacter sp. RHBSTW-00570: 2742655 | increased |
| Streptomyces sp. S1D4-14: 2594461 | increased |
| Serratia sp. LS-1: 2485839 | increased |
| Bacteria: Spirochaetes: Treponema pallidum subsp. | increased |
| pertenue str. SamoaD: 160 | |
| Arcobacter thereius LMG 24486: 544718 | increased |
| Xanthomonas perforans 91-118: 442694 | increased |
| Actinomyces sp. oral taxon 169: 712116 | increased |
| Prevotella dentalis DSM 3688: 52227 | increased |
| Flavonifractor plautii: 292800 | increased |
| Corynebacterium mycetoides: 38302 | increased |
| Actinomyces sp. oral taxon 171 str. F0337: 706438 | increased |
| Prevotella oris: 28135 | increased |
| Prevotella denticola F0289: 28129 | increased |
| Bacteroides sp. A1C1: 2528203 | increased |
| Tannerella forsythia KS16: 28112 | increased |
| Aeromonas salmonicida subsp. smithia: 645 | increased |
| Aeromonas sp. ASNIH3: 1636608 | increased |
| [Arcobacter] porcinus: 1935204 | increased |
| Cardiobacterium hominis: 2718 | increased |
| Bacteroides cellulosilyticus: 246787 | increased |
| Desulfovibrio sp. G11: 631220 | increased |
| Stenotrophomonas acidaminiphila: 128780 | increased |
| Delftia tsuruhatensis: 180282 | increased |
| Xanthomonas translucens pv. undulosa: 343 | increased |
| Stenotrophomonas nitritireducens: 83617 | increased |
| Candidatus Nanosynbacter lyticus: 2093824 | increased |
| Comamonas aquatica: 225991 | increased |
| Bacteroides heparinolyticus: 28113 | increased |
| Bacteroides fragilis YCH46: 817 | increased |
| Pseudopropionibacterium propionicum F0230a: 1750 | increased |
| Bacteroides uniformis: 820 | increased |
| Ottowia sp. oral taxon 894: 1658672 | increased |
| Bacteroides intestinalis: 329854 | increased |
| Bacteroides caccae: 47678 | increased |
| Comamonas sp. NLF-7-7: 2597701 | increased |
| Acidovorax carolinensis: 553814 | increased |
| Melaminivora sp. SC2-9: 2109913 | increased |
| Ottowia oryzae: 2109914 | increased |
| Bacteroides caecimuris: 1796613 | increased |
| Dermabacter jinjuensis: 1667168 | increased |
| Porphyromonas gingivalis W83: 837 | increased |
| Diaphorobacter polyhydroxybutyrativorans: 1546149 | increased |
| Acidovorax sp. T1: 1858609 | increased |
| Ralstonia mannitolilytica: 105219 | increased |
| Pseudomonas denitrificans (nom. rej.): 43306 | increased |
| Desulfomicrobium orale DSM 12838: 132132 | increased |
| Desulfobulbus oralis: 1986146 | increased |
| Acidovorax sp. JS42: 232721 | increased |
| Bacteroides xylanisolvens: 371601 | increased |
| Bacteroides zoogleoformans: 28119 | increased |
| Alicycliphilus denitrificans K601: 179636 | increased |
| Diaphorobacter sp. JS3050: 2735554 | increased |
| Acidovorax ebreus TPSY: 721785 | increased |

TABLE 9
The relative increased or decreased abundance for each
predictive microbe for food allergic dermatitis.

| | Increase/ |
|---|---|
| | decreased |
| | relative |
| Microbe | abundance |
| Frederiksenia canicola: 123824 | decreased |
| Avibacterium paragallinarum: 728 | decreased |
| Pasteurella dagmatis: 754 | decreased |
| Streptobacillus moniliformis DSM 12112: 34105 | decreased |
| Haemophilus haemolyticus: 726 | decreased |
| Glaesserella sp. 15-184: 2030797 | decreased |
| Conchiformibius steedae: 153493 | decreased |
| Moraxella catarrhalis BBH18: 480 | decreased |
| Moraxella cuniculi: 34061 | decreased |
| Neisseria zoodegmatis: 326523 | decreased |
| Moraxella bovoculi: 386891 | decreased |
| Neisseria animaloris: 326522 | decreased |
| Neisseria weaveri: 28091 | decreased |
| Moraxella ovis: 29433 | decreased |
| Moraxella osloensis: 34062 | decreased |
| Saccharomyces cerevisiae S288C: 4932 | decreased |
| Neisseria wadsworthii: 607711 | decreased |
| Histophilus somni 2336: 731 | decreased |
| Neisseria musculi: 1815583 | decreased |
| Fusobacterium sp. oral taxon 203: 671211 | decreased |
| Neisseria canis: 493 | decreased |
| Saccharomyces eubayanus: 1080349 | decreased |
| Capnocytophaga canimorsus Cc5: 28188 | decreased |
| Salmonella enterica subsp. salamae serovar | decreased |
| 57:z29:z42: 28901 | |
| Fusobacterium pseudoperiodonticum: 2663009 | decreased |
| Alloprevotella sp. E39: 2133944 | decreased |
| Fusobacterium hwasookii ChDC F300: 1583098 | decreased |
| Capnocytophaga sp. H4358: 1945658 | decreased |
| Pasteurella multocida subsp. septica: 747 | decreased |
| Cutibacterium acnes subsp. defendens ATCC 11828: 1747 | decreased |
| Capnocytophaga sp. H2931: 1945657 | decreased |
| Lautropia mirabilis: 47671 | decreased |
| Streptococcus dysgalactiae subsp. equisimilis RE378: 1334 | decreased |
| Parvimonas micra: 33033 | decreased |
| Neisseria shayeganii: 607712 | decreased |
| Acinetobacter johnsonii XBB1: 40214 | decreased |
| Porphyromonas asaccharolytica DSM 20707: 28123 | decreased |
| Dichelobacter nodosus VCS1703A: 870 | decreased |
| Porphyromonas cangingivalis: 36874 | decreased |
| Neisseria dentiae: 194197 | decreased |
| Streptococcus equi subsp. zooepidemicus MGCS10565: 1336 | decreased |
| Prevotella fusca JCM 17724: 589436 | decreased |
| Psychrobacter sp. PRwf-1: 349106 | decreased |
| Fusobacterium nucleatum subsp. vincentii ChDC F8: 851 | decreased |
| Leptotrichia sp. oral taxon 212: 712357 | decreased |
| Psychrobacter sp. P2G3: 1699622 | decreased |
| Lachnoanaerobaculum umeaense: 617123 | decreased |
| Psychrobacter sp. P11G5: 1699624 | decreased |
| Streptococcus canis: 1329 | decreased |
| Shigella sonnei: 624 | decreased |
| Capnocytophaga cynodegmi: 28189 | decreased |
| Yersinia pestis biovar Medievalis str. Harbin 35: 632 | decreased |
| Klebsiella sp. MPUS7: 2697371 | increased |
| Streptomyces sp. ICC4: 2099584 | increased |
| Streptomyces sp. S1D4-14: 2594461 | increased |
| Citrobacter sp. RHBSTW-00229: 2742641 | increased |
| Citrobacter sp. RHBSTW-00570: 2742655 | increased |
| Pseudomonas sp. EGD-AKN5: 1524461 | increased |
| Citrobacter sp. RHBSTW-01044: 2742678 | increased |
| Klebsiella sp. WP3-W18-ESBL-02: 2675710 | increased |
| Serratia sp. LS-1: 2485839 | increased |
| Bacteroides sp. HF-162: 2785531 | increased |
| Pseudomonas sp. FDAARGOS_761: 2545800 | increased |
| Burkholderia sp. 2002721687: 1468409 | increased |
| Xanthomonas perforans 91-118: 442694 | increased |
| Actinomyces sp. oral taxon 169: 712116 | increased |
| Corynebacterium mycetoides: 38302 | increased |
| Actinomyces sp. oral taxon 171 str. F0337: 706438 | increased |
| Xanthomonas euroxanthea: 2259622 | increased |
| Arcobacter thereius LMG 24486: 544718 | increased |
| Bacteroides sp. A1C1: 2528203 | increased |
| Treponema pedis str. T A4: 409322 | increased |
| Streptococcus intermedius JTH08: 1338 | increased |
| Flavonifractor plautii: 292800 | increased |
| Campylobacter sp. RM16192: 1660080 | increased |
| Bergeyella cardium: 1585976 | increased |
| Prevotella oris: 28135 | increased |
| Tannerella forsythia KS16: 28112 | increased |
| Stenotrophomonas acidaminiphila: 128780 | increased |
| Aeromonas salmonicida subsp. smithia: 645 | increased |
| Treponema putidum: 221027 | increased |
| Treponema denticola OTK: 158 | increased |
| Prevotella denticola F0289: 28129 | increased |
| Candidatus Nanosynbacter lyticus: 2093824 | increased |
| [Arcobacter] porcinus: 1935204 | increased |
| Treponema sp. OMZ 804: 120683 | increased |
| Lysobacter oculi: 2698682 | increased |
| Treponema sp. OMZ 838: 1539298 | increased |
| Bacteroides uniformis: 820 | increased |
| Aeromonas sp. ASNIH3: 1636608 | increased |
| Bacteroides heparinolyticus: 28113 | increased |
| Bacteroides cellulosilyticus: 246787 | increased |
| Desulfovibrio sp. G11: 631220 | increased |
| Delftia tsuruhatensis: 180282 | increased |
| Acidovorax carolinensis: 553814 | increased |
| Cardiobacterium hominis: 2718 | increased |
| Comamonas sp. NLF-7-7: 2597701 | increased |
| Stenotrophomonas nitritireducens: 83617 | increased |
| Ottowia sp. oral taxon 894: 1658672 | increased |
| Bacteroides intestinalis: 329854 | increased |
| Porphyromonas gingivalis W83: 837 | increased |
| Bacteroides caccae: 47678 | increased |
| Comamonas aquatica: 225991 | increased |
| Bacteroides fragilis YCH46: 817 | increased |
| Diaphorobacter polyhydroxybutyrativorans: 1546149 | increased |
| Dermabacter jinjuensis: 1667168 | increased |
| Melaminivora sp. SC2-9: 2109913 | increased |
| Pseudopropionibacterium propionicum F0230a: 1750 | increased |
| Ralstonia mannitolilytica: 105219 | increased |
| Ottowia oryzae: 2109914 | increased |
| Xanthomonas translucens pv. undulosa: 343 | increased |
| Pseudomonas denitrificans (nom. rej.): 43306 | increased |
| Acidovorax sp. T1: 1858609 | increased |
| Bacteroides caecimuris: 1796613 | increased |
| Alicycliphilus denitrificans K601: 179636 | increased |
| Acidovorax sp. JS42: 232721 | increased |
| Desulfomicrobium orale DSM 12838: 132132 | increased |
| Desulfobulbus oralis: 1986146 | increased |
| Acidovorax ebreus TPSY: 721785 | increased |
| Bacteroides zoogleoformans: 28119 | increased |
| Bacteroides xylanisolvens: 371601 | increased |
| Diaphorobacter sp. JS3050: 2735554 | increased |

TABLE 10
The relative increased or decreased abundance for each
predictive microbe for flea allergic dermatitis.

| | Increase/ |
|---|---|
| | decreased |
| | relative |
| Microbe | abundance |
| Frederiksenia canicola: 123824 | decreased |
| Streptobacillus moniliformis DSM 12112: 34105 | decreased |
| Avibacterium paragallinarum: 728 | decreased |
| Pasteurella dagmatis: 754 | decreased |
| Haemophilus haemolyticus: 726 | decreased |
| Glaesserella sp. 15-184: 2030797 | decreased |
| Moraxella catarrhalis BBH18: 480 | decreased |
| Neisseria weaveri: 28091 | decreased |
| Conchiformibius steedae: 153493 | decreased |
| Neisseria zoodegmatis: 326523 | decreased |
| Fusobacterium pseudoperiodonticum: 2663009 | decreased |
| Moraxella cuniculi: 34061 | decreased |
| Capnocytophaga canimorsus Cc5: 28188 | decreased |
| Fusobacterium hwasookii ChDC F300: 1583098 | decreased |
| Fusobacterium sp. oral taxon 203: 671211 | decreased |
| Neisseria animaloris: 326522 | decreased |
| Moraxella bovoculi: 386891 | decreased |
| Histophilus somni 2336: 731 | decreased |
| Neisseria wadsworthii: 607711 | decreased |
| Moraxella ovis: 29433 | decreased |
| Capnocytophaga sp. H4358: 1945658 | decreased |
| Capnocytophaga sp. H2931: 1945657 | decreased |
| Neisseria canis: 493 | decreased |
| Fusobacterium periodonticum: 860 | decreased |
| Neisseria musculi: 1815583 | decreased |
| Pasteurella multocida subsp. septica: 747 | decreased |
| Moraxella osloensis: 34062 | decreased |
| Acinetobacter johnsonii XBB1: 40214 | decreased |
| Fusobacterium nucleatum subsp. vincentii ChDC | decreased |
| F8: 851 | |
| Capnocytophaga cynodegmi: 28189 | decreased |
| Neisseria shayeganii: 607712 | decreased |
| Parvimonas micra: 33033 | decreased |
| Streptococcus dysgalactiae subsp. equisimilis | decreased |
| RE378: 1334 | |
| Leptotrichia sp. oral taxon 212: 712357 | decreased |
| Burkholderia mallei SAVP1: 13373 | decreased |
| Campylobacter sp. CFSAN093260: 2572085 | decreased |
| Campylobacter sp. CFSAN093256: 2572082 | decreased |
| Klebsiella sp. WP4-W18-ESBL-05: 2675713 | decreased |
| Dietzia sp. DQ12-45-1b: 912801 | decreased |
| Brucella abortus bv. 9 str. C68: 235 | decreased |
| Staphylococcus piscifermentans: 70258 | increased |
| Brevibacterium sp. PAMC23299: 2762330 | increased |
| Tessaracoccus lapidicaptus: 1427523 | increased |
| Pseudomonas sp. FDAARGOS_761: 2545800 | increased |
| Arcobacter thereius LMG 24486: 544718 | increased |
| Corynebacterium sanguinis: 2594913 | increased |
| Chryseobacterium gallinarum: 1324352 | increased |
| Cardiobacterium hominis: 2718 | increased |
| Clostridioides difficile R20291: 1496 | increased |
| Bacteroides sp. A1C1: 2528203 | increased |
| Stenotrophomonas nitritireducens: 83617 | increased |
| Tannerella forsythia KS16: 28112 | increased |
| Treponema pedis str. T A4: 409322 | increased |
| Flavonifractor plautii: 292800 | increased |
| Bergeyella cardium: 1585976 | increased |
| Stenotrophomonas acidaminiphila: 128780 | increased |
| Pseudopropionibacterium propionicum F0230a: 1750 | increased |
| Ottowia sp. oral taxon 894: 1658672 | increased |
| Candidatus Nanosynbacter lyticus: 2093824 | increased |
| Bacteroides cellulosilyticus: 246787 | increased |
| Xanthomonas translucens pv. undulosa: 343 | increased |
| Campylobacter sp. RM16192: 1660080 | increased |
| Prevotella denticola F0289: 28129 | increased |
| Bacteroides uniformis: 820 | increased |
| Comamonas aquatica: 225991 | increased |
| Desulfovibrio sp. G11: 631220 | increased |
| Prevotella oris: 28135 | increased |
| Melaminivora sp. SC2-9: 2109913 | increased |
| Bacteroides heparinolyticus: 28113 | increased |
| Ottowia oryzae: 2109914 | increased |
| [Arcobacter] porcinus: 1935204 | increased |
| Aeromonas sp. ASNIH3: 1636608 | increased |
| Delftia tsuruhatensis: 180282 | increased |
| Bacteroides intestinalis: 329854 | increased |
| Comamonas sp. NLF-7-7: 2597701 | increased |
| Ralstonia mannitolilytica: 105219 | increased |
| Bacteroides caccae: 47678 | increased |
| Bacteroides fragilis YCH46: 817 | increased |
| Diaphorobacter polyhydroxybutyrativorans: 1546149 | increased |
| Pseudomonas denitrificans (nom. rej.): 43306 | increased |
| Dermabacter jinjuensis: 1667168 | increased |
| Treponema denticola OTK: 158 | increased |
| Acidovorax carolinensis: 553814 | increased |
| Bacteroides xylanisolvens: 371601 | increased |
| Alicycliphilus denitrificans K601: 179636 | increased |
| Treponema phagedenis: 162 | increased |
| Treponema putidum: 221027 | increased |
| Treponema sp. OMZ 804: 120683 | increased |
| Aeromonas salmonicida subsp. smithia: 645 | increased |
| Acidovorax ebreus TPSY: 721785 | increased |
| Acidovorax sp. JS42: 232721 | increased |
| Porphyromonas gingivalis W83: 837 | increased |
| Treponema sp. OMZ 838: 1539298 | increased |
| Bacteroides caecimuris: 1796613 | increased |
| Desulfobulbus oralis: 1986146 | increased |
| Acidovorax sp. T1: 1858609 | increased |
| Bacteroides zoogleoformans: 28119 | increased |
| Desulfomicrobium orale DSM 12838: 132132 | increased |
| Diaphorobacter sp. JS3050: 2735554 | increased |

TABLE 11
The relative increased or decreased abundance for each
predictive microbe for atopic dermatitis.

| | Increase/ |
|---|---|
| | decreased |
| | relative |
| Microbe | abundance |
| Frederiksenia canicola: 123824 | decreased |
| Avibacterium paragallinarum: 728 | decreased |
| Pasteurella dagmatis: 754 | decreased |
| Streptobacillus moniliformis DSM 12112: 34105 | decreased |
| Glaesserella sp. 15-184: 2030797 | decreased |
| Haemophilus haemolyticus: 726 | decreased |
| Conchiformibius steedae: 153493 | decreased |
| Moraxella catarrhalis BBH18: 480 | decreased |
| Neisseria zoodegmatis: 326523 | decreased |
| Moraxella cuniculi: 34061 | decreased |
| Neisseria animaloris: 326522 | decreased |
| Moraxella bovoculi: 386891 | decreased |
| Neisseria weaveri: 28091 | decreased |
| Neisseria musculi: 1815583 | decreased |
| Neisseria wadsworthii: 607711 | decreased |
| Moraxella ovis: 29433 | decreased |
| Moraxella osloensis: 34062 | decreased |
| Histophilus somni 2336: 731 | decreased |
| Neisseria canis: 493 | decreased |
| Cutibacterium acnes subsp. defendens ATCC | decreased |
| 11828: 1747 | |
| Pseudomonas sp. TKP: 1415630 | decreased |
| Saccharomyces cerevisiae S288C: 4932 | decreased |
| Fusobacterium pseudoperiodonticum: 2663009 | decreased |
| Neisseria dentiae: 194197 | decreased |
| Capnocytophaga sp. H4358: 1945658 | decreased |
| Capnocytophaga sp. H2931: 1945657 | decreased |
| Capnocytophaga canimorsus Cc5: 28188 | decreased |
| Neisseria shayeganii: 607712 | decreased |
| Pasteurella multocida subsp. septica: 747 | decreased |
| Actinomyces oris: 544580 | decreased |
| Parvimonas micra: 33033 | decreased |
| Psychrobacter sp. PRwf-1: 349106 | decreased |
| Streptomyces sp. GBA 94-10: 1218177 | increased |
| [Brevibacterium] flavum ZL-1: 92706 | increased |
| Pseudomonas sp. EGD-AKN5: 1524461 | increased |
| Tessaracoccus lapidicaptus: 1427523 | increased |
| Xanthomonas perforans 91-118: 442694 | increased |
| Arcobacter thereius LMG 24486: 544718 | increased |
| Capnocytophaga stomatis: 1848904 | increased |
| Streptococcus intermedius JTH08: 1338 | increased |
| Porphyromonas crevioricanis: 393921 | increased |
| Chryseobacterium gallinarum: 1324352 | increased |
| Xanthomonas translucens pv. undulosa: 343 | increased |
| Prevotella oris: 28135 | increased |
| Bergeyella cardium: 1585976 | increased |
| Aeromonas salmonicida subsp. smithia: 645 | increased |
| Candidatus Nanosynbacter lyticus: 2093824 | increased |
| Clostridioides difficile R20291: 1496 | increased |
| Tannerella forsythia KS16: 28112 | increased |
| Stenotrophomonas acidaminiphila: 128780 | increased |
| Dermabacter jinjuensis: 1667168 | increased |
| Treponema sp. OMZ 804: 120683 | increased |
| Stenotrophomonas nitritireducens: 83617 | increased |
| Campylobacter rectus: 203 | increased |
| Porphyromonas gingivalis W83: 837 | increased |
| Comamonas aquatica: 225991 | increased |
| Delftia tsuruhatensis: 180282 | increased |
| Treponema sp. OMZ 838: 1539298 | increased |
| Pseudomonas denitrificans (nom. rej.): 43306 | increased |
| Ottowia sp. oral taxon 894: 1658672 | increased |
| Prevotella denticola F0289: 28129 | increased |
| Comamonas sp. NLF-7-7: 2597701 | increased |
| Desulfovibrio sp. G11: 631220 | increased |
| Bacteroides sp. A1C1: 2528203 | increased |
| Bacteroides caccae: 47678 | increased |
| Melaminivora sp. SC2-9: 2109913 | increased |
| Diaphorobacter polyhydroxybutyrativorans: 1546149 | increased |
| Bacteroides intestinalis: 329854 | increased |
| Bacteroides fragilis YCH46: 817 | increased |
| Bacteroides uniformis: 820 | increased |
| [Arcobacter] porcinus: 1935204 | increased |
| Bacteroides cellulosilyticus: 246787 | increased |
| Acidovorax carolinensis: 553814 | increased |
| Acidovorax sp. T1: 1858609 | increased |
| Ralstonia mannitolilytica: 105219 | increased |
| Pseudopropionibacterium propionicum F0230a: 1750 | increased |
| Ottowia oryzae: 2109914 | increased |
| Bacteroides heparinolyticus: 28113 | increased |
| Alicycliphilus denitrificans K601: 179636 | increased |
| Bacteroides caecimuris: 1796613 | increased |
| Acidovorax ebreus TPSY: 721785 | increased |
| Diaphorobacter sp. JS3050: 2735554 | increased |
| Desulfomicrobium orale DSM 12838: 132132 | increased |
| Bacteroides zoogleoformans: 28119 | increased |
| Bacteroides xylanisolvens: 371601 | increased |
| Desulfobulbus oralis: 1986146 | increased |

TABLE 12
The relative increased or decreased abundance for each predictive
microbe for enviornmental allergic dermatitis.

| | Increase/ |
|---|---|
| | decreased |
| | relative |
| Microbe | abundance |
| Frederiksenia canicola: 123824 | decreased |
| Streptobacillus moniliformis DSM 12112: 34105 | decreased |
| Pasteurella dagmatis: 754 | decreased |
| Avibacterium paragallinarum: 728 | decreased |
| Glaesserella sp. 15-184: 2030797 | decreased |
| Haemophilus haemolyticus: 726 | decreased |
| Conchiformibius steedae: 153493 | decreased |
| Moraxella catarrhalis BBH18: 480 | decreased |
| Moraxella bovoculi: 386891 | decreased |
| Moraxella cuniculi: 34061 | decreased |
| Neisseria zoodegmatis: 326523 | decreased |
| Histophilus somni 2336: 731 | decreased |
| Neisseria weaveri: 28091 | decreased |
| Moraxella ovis: 29433 | decreased |
| Fusobacterium sp. oral taxon 203: 671211 | decreased |
| Neisseria animaloris: 326522 | decreased |
| Moraxella osloensis: 34062 | decreased |
| Capnocytophaga canimorsus Cc5: 28188 | decreased |
| Fusobacterium pseudoperiodonticum: 2663009 | decreased |
| Saccharomyces cerevisiae S288C: 4932 | decreased |
| Parvimonas micra: 33033 | decreased |
| Fusobacterium hwasookii ChDC F300: 1583098 | decreased |
| Capnocytophaga sp. H4358: 1945658 | decreased |
| Capnocytophaga sp. H2931: 1945657 | decreased |
| Streptococcus dysgalactiae subsp. equisimilis | decreased |
| RE378: 1334 | |
| Neisseria wadsworthii: 607711 | decreased |
| Neisseria musculi: 1815583 | decreased |
| Neisseria canis: 493 | decreased |
| Leptotrichia sp. oral taxon 212: 712357 | decreased |
| Streptococcus oralis subsp. tigurinus: 1303 | decreased |
| Pasteurella multocida subsp. septica: 747 | decreased |
| Lachnoanaerobaculum umeaense: 617123 | decreased |
| Porphyromonas asaccharolytica DSM 20707: 28123 | decreased |
| Fungi: Basidiomycota: Malassezia restricta: 76775 | decreased |
| Fusobacterium nucleatum subsp. vincentii ChDC F8: 851 | decreased |
| Dichelobacter nodosus VCS1703A: 870 | decreased |
| Streptococcus equi subsp. zooepidemicus | decreased |
| MGCS10565: 1336 | |
| Porphyromonas cangingivalis: 36874 | decreased |
| Saccharomyces eubayanus: 1080349 | decreased |
| Alloprevotella sp. E39: 2133944 | decreased |
| Acinetobacter johnsonii XBB1: 40214 | decreased |
| Streptococcus canis: 1329 | decreased |
| Capnocytophaga cynodegmi: 28189 | decreased |
| Campylobacter sp. CCUG 57310: 2517362 | decreased |
| Fusobacterium necrophorum subsp. necrophorum: 859 | decreased |
| Neisseria shayeganii: 607712 | decreased |
| Acinetobacter lwoffii WJ10621: 28090 | decreased |
| Streptococcus pseudoporcinus: 361101 | decreased |
| Klebsiella sp. MPUS7: 2697371 | increased |
| Enterobacter sp. CRENT-193: 2051905 | increased |
| Streptomyces sp. S1D4-14: 2594461 | increased |
| Klebsiella sp. WP3-W18-ESBL-02: 2675710 | increased |
| Citrobacter freundii complex sp. CFNIH3: 2077147 | increased |
| Pseudomonas sp. EGD-AKN5: 1524461 | increased |
| Salmonella sp. S13: 2686305 | increased |
| Shigella boydii Sb227: 621 | increased |
| Aeromonas sp. ASNIH7: 1920107 | increased |
| Pseudomonas sp. FDAARGOS_761: 2545800 | increased |
| Xanthomonas perforans 91-118: 442694 | increased |
| Actinomyces sp. oral taxon 171 str. F0337: 706438 | increased |
| Flavonifractor plautii: 292800 | increased |
| Xanthomonas euroxanthea: 2259622 | increased |
| Corynebacterium mycetoides: 38302 | increased |
| Arcobacter thereius LMG 24486: 544718 | increased |
| Actinomyces sp. oral taxon 169: 712116 | increased |
| Tannerella forsythia KS16: 28112 | increased |
| Pseudomonas sp. TKP: 1415630 | increased |
| Prevotella oris: 28135 | increased |
| Bacteroides sp. A1C1: 2528203 | increased |
| Aeromonas salmonicida subsp. smithia: 645 | increased |
| [Arcobacter] porcinus: 1935204 | increased |
| Bacteroides cellulosilyticus: 246787 | increased |
| Prevotella denticola F0289: 28129 | increased |
| Treponema sp. OMZ 838: 1539298 | increased |
| Treponema sp. OMZ 804: 120683 | increased |
| Bacteroides heparinolyticus: 28113 | increased |
| Cardiobacterium hominis: 2718 | increased |
| Desulfovibrio sp. G11: 631220 | increased |
| Bacteroides fragilis YCH46: 817 | increased |
| Lysobacter oculi: 2698682 | increased |
| Bacteroides uniformis: 820 | increased |
| Comamonas sp. NLF-7-7: 2597701 | increased |
| Dermabacter jinjuensis: 1667168 | increased |
| Ottowia sp. oral taxon 894: 1658672 | increased |
| Bacteroides intestinalis: 329854 | increased |
| Bacteroides caccae: 47678 | increased |
| Comamonas aquatica: 225991 | increased |
| Xanthomonas translucens pv. undulosa: 343 | increased |
| Porphyromonas gingivalis W83: 837 | increased |
| Melaminivora sp. SC2-9: 2109913 | increased |
| Pseudopropionibacterium propionicum F0230a: 1750 | increased |
| Stenotrophomonas acidaminiphila: 128780 | increased |
| Ottowia oryzae: 2109914 | increased |
| Aeromonas sp. ASNIH3: 1636608 | increased |
| Delftia tsuruhatensis: 180282 | increased |
| Diaphorobacter polyhydroxybutyrativorans: 1546149 | increased |
| Stenotrophomonas nitritireducens: 83617 | increased |
| Acidovorax carolinensis: 553814 | increased |
| Desulfomicrobium orale DSM 12838: 132132 | increased |
| Ralstonia mannitolilytica: 105219 | increased |
| Alicycliphilus denitrificans K601: 179636 | increased |
| Bacteroides caecimuris: 1796613 | increased |
| Acidovorax sp. T1: 1858609 | increased |
| Pseudomonas denitrificans (nom. rej.): 43306 | increased |
| Desulfobulbus oralis: 1986146 | increased |
| Bacteroides xylanisolvens: 371601 | increased |
| Acidovorax sp. JS42: 232721 | increased |
| Acidovorax ebreus TPSY: 721785 | increased |
| Bacteroides zoogleoformans: 28119 | increased |
| Diaphorobacter sp. JS3050: 2735554 | increased |

### CONCLUSION

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

1. A method for screening, detecting, and/or preventing dermatologic disease in cats, the method comprising:
obtaining an oral microbial profile for a cat, the oral microbial profile comprising one or more microbial species present in an oral sample from the cat and a quantity or abundance of the one or more microbial species in the oral sample;
comparing the oral microbial profile to information in a database that identifies weighted correlations between:
(i) occurrence and/or prevalence of one or more dermatologic diseases in cats; and
(ii) presence and/or abundance of various microbial species in oral microbiomes of cats, wherein the various microbial species comprise the one or more microbial species in the oral sample;
generating a risk score indicating a likelihood that the cat has the one or more dermatologic diseases based on one or more matches between the oral microbial profile and the information in the database; and
categorizing the cat as developing the one or more dermatologic diseases when the risk score meets or exceeds a predetermined threshold and, optionally, prescribing a therapeutic treatment protocol suitable for treating, mitigating, or preventing the development, advancement, or recurrence of the one or more dermatologic diseases when the risk score meets or exceeds a predetermined threshold.

2. (canceled)

3. The method of claim 1, wherein obtaining the oral microbial profile for the cat comprises:
obtaining nucleic acid sequence data corresponding to microbial nucleic acid obtained from the oral sample;
analyzing the nucleic acid sequence data to identify the one or more microbial species present in the oral sample and, optionally, quantifying the one or more microbial species; and
generating the oral microbial profile for the cat based on the identified and, optionally, quantified one or more microbial species.

4. The method of claim 3, wherein obtaining the microbial nucleic acid sequence data comprises:
sequencing microbial nucleic acid from the oral sample; and, optionally,
isolating the microbial nucleic acid from the oral sample.

5. The method of claim 4, wherein isolating the microbial nucleic acid from the oral sample comprises:
performing heat treatment on the oral sample; and
performing magnetic SPRI beads-based nucleic acid extraction on the heat-treated oral sample, with or without the addition of protein digesting reagents and detergents, to extract the microbial nucleic acid from the oral sample.

6. The method of claim 3, wherein analyzing the microbial nucleic acid sequence data comprises one or more of:
demultiplexing the nucleic acid sequence data;
trimming the nucleic acid sequence data;
mapping one or more unmapped reads onto a reference genome of the cat and/or onto existing microbial reference genomes;
classifying one or more reads as feline from the nucleic acid sequence data after mapping;
classifying one or more reads as microbial from the nucleic acid sequence data after mapping;
quantifying the one or more microbial reads;
transforming the quantified one or more microbial reads to account for sequence coverage biases using methods such as pairwise log ratio transformation; and
comparing compositional abundance patterns in the transformed one or more microbial reads against compositional abundance patterns in transformed data in a reference database comprising samples from cats that do not suffer from dermatologic diseases, as well as samples from cats that suffer from specific dermatologic diseases.

7. The method of claim 1, wherein comparing the oral microbial profile to the information in the database comprises one or more of:
calculating the abundance of the one or more microbial species in the oral sample;
identifying the one or more microbial species in the oral sample; and
comparing the abundance of the identified one or more microbial species in the oral sample to the presence and/or abundance of various microbial species in the oral microbiome of cats.

8. The method of claim 1, wherein generating the risk score comprises one or more of:
identifying one or more similarities between compositional abundances of the one or more microbial species in the oral sample and compositional abundances of various microbial species in the oral microbiome of cats contained in the database;
identifying one or more matches between the identity of the one or more microbial species in the oral sample and the presence of various microbial species in the oral microbiome of cats contained in the database;
quantifying the identified one or more similarities between the compositional abundances of the one or more microbial species in the oral sample and the compositional abundances of the one or more microbial species in the oral microbiome of cats contained in the database; and
identifying a presence of one or more predictive microbial species in the oral sample.

9. The method of claim 1, wherein the one or more dermatologic diseases is selected from the group consisting of atopic dermatitis, food allergic dermatitis, flea allergic dermatitis, and environmental allergic dermatitis.

10. The method of claim 1 further comprising:
generating a report presenting (i) the risk score, (ii) an indication of developing the one or more dermatologic diseases when the risk score meets or exceeds the predetermined threshold, (iii) a timing recommendation, (iv) optionally, one or more at home practices to improve dermatologic health, (v) optionally, one or more diagnostic steps to diagnose the one or more dermatologic diseases when the risk score meets or exceeds the predetermined threshold, and (vi) optionally, a prescription for the therapeutic treatment protocol; and, optionally,
communicating the generated report electronically to an owner of the cat and/or their veterinarian.

11. (canceled)

12. A computer system configured to indicate or predict dermatologic disease in cats, the computer system comprising:
one or more processors; and
one or more computer-readable hardware storage devices having stored thereon instructions that are executable by the one or more processors to configure the computer system to:
receive microbial nucleic acid sequence data corresponding to microbial nucleic acid obtained from an oral sample taken from a cat;
analyze the microbial nucleic acid sequence data to identify one or more microbial species present in the oral sample and quantify the one or more microbial species;
generate an oral microbial profile for the cat based on the identified one or more microbial species and their respective abundances;
compare the oral microbial profile to information in a database that identifies weighted correlations between:
(i) occurrence and/or prevalence of one or more dermatologic diseases in cats; and
(ii) presence and/or abundance of various microbial species in oral microbiomes of cats, wherein the various microbial species comprise the one or more microbial species in the oral sample;
identify one or more matches between the oral microbial profile and the information in the database;
generate a risk score indicating a likelihood that the cat has the one or more dermatologic diseases based on the one or more matches between the oral microbial profile and the information in the database; and, optionally,
diagnose the cat as “developing” the one or more dermatologic diseases when the risk score meets or exceeds a predetermined threshold,
prescribe a therapeutic treatment protocol suitable for treating or preventing the one or more dermatologic diseases when the risk score meets or exceeds the predetermined threshold,
generate a report indicating (i) the risk score, (ii) an indication of developing the one or more dermatologic diseases when the risk score meets or exceeds the predetermined threshold, (iii) a timing recommendation, (iv) optionally, one or more at home practices to improve dermatologic health, (v) optionally, one or more diagnostic steps to diagnose the one or more dermatologic diseases when the risk score meets or exceeds the predetermined threshold, and (vi) a prescription for the therapeutic treatment protocol, and/or
communicate the generated report electronically to an owner of the cat and/or their veterinarian.

13. The computer system of claim 12, wherein the instructions further configure the computer system to map one or more unmapped sequence reads to a cat reference genome and/or map one or more sequence reads to microbial reference genomes and, optionally, classify the reads as microbial or feline.

14. The computer system of claim 13, wherein the instructions further configure the computer system to identify at least one unmapped sequence read of metagenomic sequence data and, optionally, classify the at least one unmapped read.

15. The computer system of claim 13, wherein feline oral microbiome samples having fewer than 10,000 classified microbial reads or more than 500,000 classified microbial reads are excluded from the comparison of the oral microbial profile for the cat against a database of defined microbial profiles.

16. The computer system of claim 12, wherein the instructions further configure the computer system to calculate an abundance of the one or more microbial species present in the oral sample.

17. The computer system of claim 16, wherein the abundance of the specific one or more microbial species present in the oral sample correlates to whether the specific one or more microbial species is a predictive microbial species for the specific dermatologic disease.

18. The computer system of claim 16, wherein the instructions further configure the computer system to perform a pairwise log ratio comparison of the microbial abundance of the cat's oral sample against the information in the database.

19. The system of claim 18, wherein a specific one or more microbial species is a predictive microbial species when 50% or more of the maximum possible pairwise log ratio comparisons involving the specific microbe are significantly different when compared between a disease and a control cohort.

20.-42. (canceled)

43. A method for predicting the development of a dermatologic or respiratory disease in a cat, the method comprising:
obtaining an oral sample from a cat, the oral sample containing one or more microbial species;
isolating, from the oral sample, microbial nucleic acid of the one or more microbial species;
obtaining microbial nucleic acid sequence data corresponding to the microbial nucleic acid;
analyzing the microbial nucleic acid sequence data to identify one or more microbial species present in the oral sample and, optionally, quantifying the one or more microbial species;
generating an oral microbial profile for the cat based on the identified and, optionally, quantified one or more microbial species, the oral microbial profile comprising the one or more microbial species and, optionally, a quantity or relative abundance of the one or more microbial species in the oral sample;
comparing the oral microbial profile to information in a database that identifies weighted correlations between:
(i) occurrence and/or prevalence of one or more dermatologic or respiratory diseases in cats; and
(ii) presence and/or abundance of various microbial species in the oral microbiome of animals in the classification of the cat, wherein the various microbial species comprise the one or more microbial species in the oral sample;
generating a risk score indicating a likelihood of the cat developing the one or more dermatologic or respiratory diseases based on one or more matches between the oral microbial profile and the information in the database; and
indicating the cat as developing the one or more dermatologic or respiratory diseases when the risk score meets or exceeds a predetermined threshold.

44. The method of claim 43, wherein the oral microbial profile for the cat further comprises a percentage of gram-positive microbes and wherein the various microbial species in the oral microbiome of animals in the classification of the cat further comprises a percentage of gram-positive microbes.
